(19) United States
(12) Patent Application Publication
(10) Pub. No.: US 2016/0130257 A1
(43) Pub. Date: May 12, 2016

(54) Macrocyclic proteasome inhibitors

(71) Applicant: BROWN UNIVERSITY, Providence, RI (US)

(72) Inventor: Jason K. Sello, Providence, RI (US)

(73) Assignee: BROWN UNIVERSITY, Providence, RI (US)

(21) Appl. No.: 14/935,544

(22) Filed: Nov. 9, 2015

Related U.S. Application Data
(63) Continuation of application No. PCT/US2014/037468, filed on May 9, 2014.
(60) Provisional application No. 61/821,421, filed on May 9, 2013.

(51) Int. Cl. C07D 403/14; G01N 33/573; A61K 45/06; A61K 31/405; C07D 403/06

(52) U.S. Cl. CPC C07D 403/14; A61K 31/405; C07D 403/06; A61K 45/06; G01N 33/573; G01N 2500/10; G01N 2333/952

(57) ABSTRACT

Compositions are provided for selectively inhibiting bacterial proteasome. Also provided are methods for assaying the compositions, methods for treating bacterial infection, and methods for inducing apoptosis in tumor cells using the compositions.

### RELATED APPLICATIONS

This application claims the benefit of U.S. provisional application Ser. No. 61/821,421 filed May 9, 2013 entitled, “Macrocyclic proteasome inhibitors” inventor Jason Sello, the contents of which are incorporated herein by reference in its entirety.

### TECHNICAL FIELD

Compositions for selectively inhibiting bacterial proteasomes compared to mammalian proteasome, methods for synthesizing assaying the compositions, and methods for treating bacterial infection using the compositions are provided.

### BACKGROUND

Proteases are desirable therapeutic targets for the treatment of diseases, and can be inhibited by pharmacologically attractive compounds. Therapeutic compounds for the treatment of several human diseases, function by targeting proteases including hypertension, type 2 diabetes, multiple myeloma, and infection by HIV and hepatitis C. (Raju et al. 2012, Nature Reviews Drug Discovery, vol. 11 777-789). However, proteases have not been successful targets for the treatment of bacterial infections. (Raju et al. 2012).

Infection by Mycobacterium tuberculosis is a global health emergency due to the rise in diseases caused by multi-drug resistant bacteria, and interaction between infection by M. tuberculosis and HIV (Corbett et al. Arch. Intern. Med. 2003, vol. 163, 1009-1021). As drug resistance becomes established for a growing number of human pathogens, discovery of novel therapeutic targets and novel antimicrobial agents that inhibit these targets becomes urgent.

Proteasomes are protein complexes present in eukaryotes, which degrade and remove abnormal and misfolded proteins. Proteasomes are found also in archaebacteria and some bacteria. In addition to removal of misfolded proteins, proteasomes play a role also in several physiological processes such as cell cycle regulation, cell differentiation, and response to stress. Therefore, proteasomes are an attractive target for drug development

The proteasome of M. tuberculosis is essential for virulence and survival under the conditions of nitric oxide stress which results from mounting of an immune response by the host. The macrophages of the host produce nitric oxide and other reactive nitrogen intermediates in response to the infection (Darwin et al. Science, 2003, vol. 302, 1963-1966). Proteasome inhibitors that target the human proteasome have been developed as drugs to treat human diseases, such as the anti-cancer drug Bortezomib (Velcade®) used to treat multiple myeloma. Bortezomib inhibits both bacterial and human proteasomes. Proteasome activity is needed for class I major histocompatibility complex antigen presentation during an immune response (Hughes et al., J. Exp. Med. 1996, vol. 183, 1545-1552).

There is a need for proteasome inhibitors that selectively inhibit the bacterial proteasome compared to the human proteasome.

### SUMMARY

Embodiments of the invention herein provide compositions that selectively inhibit the bacterial proteasome compared to the human proteasome.

An embodiment of the invention provides a compound having the core formula:

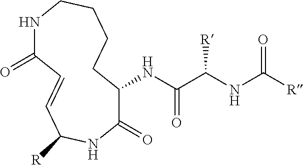

such that:

the macrocyclic ring of the core is the macrocyclic ring of syringolin B,

R is covalently attached to a macrocycle and selected from the group consisting of benzyl, alkyl substituted benzyl, halogen substituted benzyl, CF₃ substituted benzyl, indole, CH₂-indole, pyrrole, CH₂-pyrrole, imidazole, CH₂-imidazole, pyrazole, CH₂-pyrazole, pyridine, CH₂-pyridine, pyrazine, CH₂-pyrazine, pyrimidine, CH₂-pyrimidine, pyridazine, CH₂-pyridazine, heterocyclic aromatic, fused aromatic with a linked CH₂, and a fused aromatic without a linked CH₂;

R′ is selected from the group consisting of Hyrdrogen, alkyl, alkyne, alkane, alkene, benzyl, alkyl substituted benzyl, halogen substituted benzyl, CF₃ substituted benzyl, indole, CH₂-indole, pyrrole, CH₂-pyrrole, imidazole, CH₂-imidazole, pyrazole, CH₂-pyrazole, pyridine, CH₂-pyridine, pyrazine, CH₂-pyrazine, pyrimidine, CH₂-pyrimidine, pyridazine, CH₂-pyridazine, heterocyclic aromatic, fused aromatic with a linked CH₂, fused aromatic without a linked CH₂ isopropyl, CH₂CH(CH₃)₂, CH(CH₃)CH₂CH₃, CH₂CH₂—S—CH₃, CH₂SH, arginine side chain, and lysine side chain; and.

R″ is a substituted urea or an amide and the substitution is selected from the group consisting of an alkyl, an amine, an aryl, an amino acid, and a fatty acid.

In related embodiments, the macrocycle ring includes an additional unsaturated carbon-carbon bond. According to other embodiments, R″ is a substituted amino acid for example R″ is NHC(CH₃)₂COOR¹. In a related embodiment, R¹ is selected from the group consisting of methyl, ethyl, isopropyl and tertiary butyl.

In related embodiments, R¹ is a fused aromatic and is selected from the group consisting of: pentalene, indene, naphthalene, azulene, as-indacene, s-indacene, biphenylene, acenapthylene, fluorene, phenalene, heptalene, and phenanthrene. According to other embodiments of the compound, R″ is an alkyl substituted amine of length C₅-C_{15.} For example, the alkyl substituted amine is of length C₉-C₁₁. A heterocyclic compound is for example, furan, thiophene, pyran, oxazine, thiazine, benzofuran, and the like.

In related embodiments the invention provides compounds in which the macrocyclic ring of the core is the ring found in syringolin A, and R, R′, and R″ have substitutions similar to compounds in which the macrocyclic ring of the core is the ring of syringolin B.

A related embodiment of the invention provides a pharmaceutical composition including the compound as an active ingredient and a pharmaceutically acceptable carrier, salt, or buffer. The compound is an active ingredient to be used in a pharmaceutical composition as an antifungal, an antibacterial, an anticancer or an antiviral agent.

Another embodiment of the invention provides a method for inhibiting proteasome activity in a cell including contacting the cell with a compound of the core formula

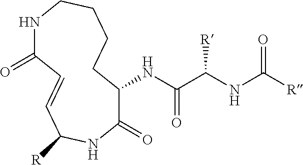

such that:

the macrocyclic ring of the core is the macrocyclic ring of syringolin B,

R is covalently attached to a macrocycle and selected from the group consisting of benzyl, alkyl substituted benzyl, halogen substituted benzyl, CF₃ substituted benzyl, indole, CH₂-indole, pyrrole, CH₂-pyrrole, imidazole, CH₂-imidazole, pyrazole, CH₂-pyrazole, pyridine, CH₂-pyridine, pyrazine, CH₂-pyrazine, pyrimidine, CH₂-pyrimidine, pyridazine, CH₂-pyridazine, heterocyclic aromatic, fused aromatic with a linked CH₂, and a fused aromatic without a linked CH₂;

R′ is selected from the group consisting of Hyrdrogen, alkyl, alkyne, alkane, alkene, benzyl, alkyl substituted benzyl, halogen substituted benzyl, CF₃ substituted benzyl, indole, CH₂-indole, pyrrole, CH₂-pyrrole, imidazole, CH₂-imidazole, pyrazole, CH₂-pyrazole, pyridine, CH₂-pyridine, pyrazine, CH₂-pyrazine, pyrimidine, CH₂-pyrimidine, pyridazine, CH₂-pyridazine, heterocyclic aromatic, fused aromatic with a linked CH₂, fused aromatic without a linked CH₂ isopropyl, CH₂CH(CH₃)₂, CH(CH₃)CH₂CH₃, CH₂CH₂—S—CH₃, CH₂SH, arginine side chain, and lysine side chain; and,

R″ is a substituted urea or an amide and the substitution is selected from the group consisting of an alkyl, an amine, an aryl, an amino acid, and a fatty acid; and,

analyzing amount of inhibition of the proteasome activity of the cell. In related embodiments the invention provides a method for inhibiting proteasome activity in a cell with a compound in which the macrocyclic ring of the core is the ring found in syringolin A, and R, R′, and R″ have substitutions similar to compounds in which the macrocyclic ring of the core is the ring of syringolin B. In a related embodiment of the method the cell is a bacterial cell. In another related embodiment of the method the cell is a eukaryotic cell. According to another embodiment of the method, analyzing inhibition of the proteasome activity includes detecting inhibition of at least one type of proteasomal activity selected from caspase-like activity, trypsin-like activity, and chymotrypsin-like activity. For example, detecting inhibition of the at least one type of proteasomal activity is performed using a small molecule substrate for the respective proteasomal activity.

In related embodiments, the macrocycle ring includes an additional unsaturated carbon-carbon bond. According to other embodiments, R″ is a substituted amino acid for example R″ is NHC(CH₃)₂COOR¹. In a related embodiment, R′ is selected from the group consisting of methyl, ethyl, isopropyl and tertiary butyl.

In related embodiments, R¹ is a fused aromatic and is selected from the group consisting of: pentalene, indene, naphthalene, azulene, as-indacene, s-indacene, biphenylene, acenapthylene, fluorene, phenalene, heptalene, and phenanthrene. According to other embodiments of the compound, R″ is an alkyl substituted amine of length C₅-C₁₅. For example, the alkyl substituted amine is of length C₉-C₁₁. A heterocyclic compound is for example, furan. thiophene, pyran, oxazine, thiazine, benzofuran, and the like.

According to one aspect of the method, analyzing further includes observing inhibition of proteasome activity in a Mycobacterium tuberculosis cell at an amount which is greater than an amount of inhibition of a human cell proteasome activity by at least: about 2-fold, about 5-fold, about 10-fold, about 20-fold, about 30-fold, about 40-fold, or about 50-fold. According to another aspect of the method, analyzing further includes observing inhibition of proteasome activity in a Streptomyces coelicolor cell at an amount which is greater than an amount of inhibition of a human cell proteasome activity by at least: about 2-fold, about 5-fold, about 10-fold, about 20-fold, about 30-fold, about 40-fold, or about 50-fold, such that the Streptomyces coelicolor cell is a surrogate for a Mycobacterium tuberculosis cell. Streptomyces coelicolor is a non-pathogenic actinobacterial model organism.

Another embodiment of the invention provides a method for screening analogs or derivatives of syringolin which inhibit M. tuberculosis proteasome activity, the method including: culturing indicator cells with the analogs or derivatives of syringolin; and, isolating and measuring amount of mRNA of protein non-heme chloroperoxidase, such that an increase in the amount of the non-heme chloroperodixase mRNA indicates inhibition of proteasome activity in the cells. For example, the non-heme chloroperoxidase is SCO0465. For example, the strains of indicator cells are selected from the group of Streptomyces coelicolor, S. griseus, S. rimosus, S. clavuligerus, S. alboniger, S. venezuelae, S. avermitilis, S. fradiae, S. lincolnensis, S. roseosporus, S. platensis, and S. verticillus.

The invention in another embodiment provides a method of treating a mammalian subject for infection by a pathogen, the method including administering to the subject a compound of the core formula:

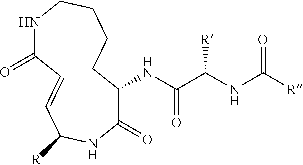

in an amount effective for inhibiting proteasome activity in the subject such that:

the macrocyclic ring of the core is the macrocyclic ring of syringolin B,

R is covalently attached to a macrocycle and selected from the group consisting of benzyl, alkyl substituted benzyl, halogen substituted benzyl, CF₃ substituted benzyl, indole, CH₂-indole, pyrrole, CH₂-pyrrole, imidazole, CH₂-imidazole, pyrazole, CH₂-pyrazole, pyridine, CH₂-pyridine, pyrazine, CH₂-pyrazine, pyrimidine, CH₂-pyrimidine, pyridazine, CH₂-pyridazine, heterocyclic aromatic, fused aromatic with a linked CH₂, and a fused aromatic without a linked CH₂;

R′ is selected from the group consisting of Hyrdrogen, alkyl, alkyne, alkane, alkene, benzyl, alkyl substituted benzyl, halogen substituted benzyl, CF₃ substituted benzyl, indole, CH₂-indole, pyrrole, CH₂-pyrrole, imidazole, CH₂-imidazole, pyrazole, CH₂-pyrazole, pyridine, CH₂-pyridine, pyrazine, CH₂-pyrazine, pyrimidine, CH₂-pyrimidine, pyridazine, CH₂-pyridazine, heterocyclic aromatic, fused aromatic with a linked CH₂, fused aromatic without a linked CH₂ isopropyl, CH2CH(CH₃)₂, CH(CH₃)CH₂CH₃, CH₂CH₂—S—CH₃, CH₂SH, arginine side chain, and lysine side chain; and,

R″ is a substituted urea or an amide and the substitution is selected from the group consisting of an alkyl, an amine, an aryl, an amino acid, and a fatty acid.

In related embodiments, the macrocycle ring includes an additional unsaturated carbon-carbon bond. According to other embodiments, R″ is a substituted amino acid for example R″ is NHC(CH₃)₂COOR¹. In a related embodiment, R¹ is selected from the group consisting of methyl, ethyl, isopropyl and tertiary butyl.

In related embodiments, R¹ is a fused aromatic and is selected from the group consisting of: pentalene, indene, naphthalene, azulene, as-indacene, s-indacene, biphenylene, acenapthylene, fluorene, phenalene, heptalene, and phenanthrene. According to other embodiments of the compound, R″ is an alkyl substituted amine of length C₅-C₁₅. For example, the alkyl substituted amine is of length C₉-C₁₁. A heterocyclic compound is for example, furan, thiophene, pyran, oxazine, thiazine, benzofuran, and the like.

In related embodiments the invention provides a method for treating a mammalian subject for infection by a pathogen with a compound in which the macrocyclic ring of the core is the ring found in syringolin A, and R, R′, and R″ have substitutions similar to compounds in which the macrocyclic ring of the core is the ring of syringolin B.

In related embodiments of the method for treating a mammalian subject, further includes treating the subject for pathogen which is selected from the group consisting of M. tuberculosis, M. leprae, M. avium, M. avium paratuberculosis, Nocardia cyriacigeorgica, N. farcinica, N. abscessus, N. asteroides, N. brasiliensis, N. nova, N. otitidiscaviarum, N. paucivorans, N. pseudobrasiliensis, N. transvalensis, N. veteran, N. wallacei, N. africana, N. anaemiae, N. araoensis, N. arthritidis, N. asiatica, N. beijingensis, N. blacklockiae, N. brevicatena, N. carnea, N. concavca, N. corynebacteroides, N. elegans, N. exalbida, N. higoensis, N. ignorata, N. inohanensis, Corynebacterium pseudotuberculosis C. renale, C. cystidis, C. pilosum, C. diphtheria and C. bovis. According to related embodiments providing methods for treating a mammalian subject, further includes measuring inhibition of the proteasome activity in the subject include inhibiting proteasome activity of M. tuberculosis in the subject, thereby inhibiting survival of the M. tuberculosis under the conditions of nitrooxidative stress that exist in vivo during infection. Related embodiments of the method further include administering an additional therapeutic agent such as isoniazid, rifampin, ethambutol, pyrazinamide, ethionamide, cycloserine, p-aminosalicyclic acid, clofazimine, amoxixillin/clavulanic acid, clarithomysin, rifabutin, thiacetazone, fluoroquinolones, and aminoglycosides and the like.

### BRIEF DESCRIPTION OF THE DRAWINGS

FIG. 1 is a schematic diagram showing targets of common antibacterial drugs and their points of inhibition.

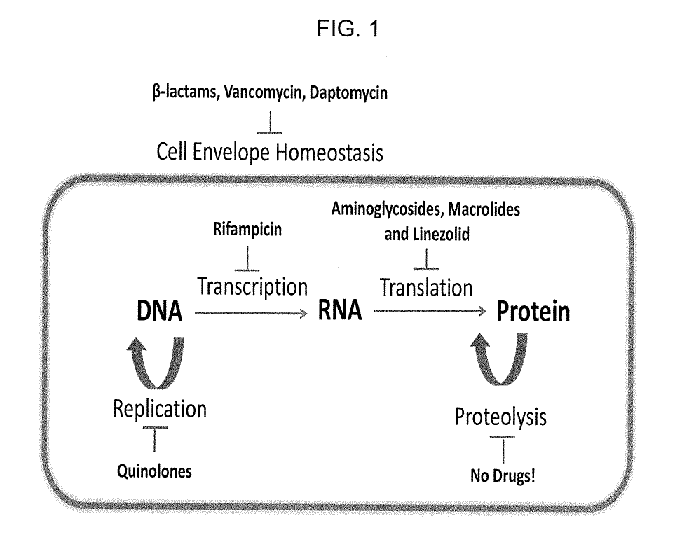

FIG. 2 is a diagram comparing protease subunits of bacterial and eukaryotic proteasomes.

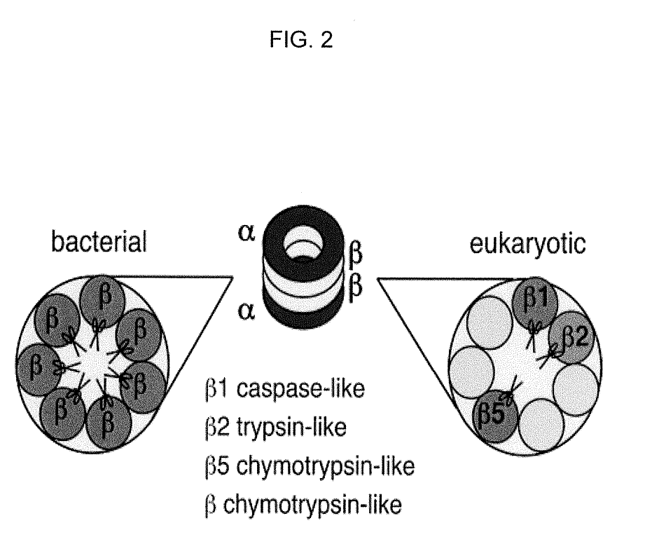

FIG. 3 is a schematic diagram showing recognition, de-ubiquitylation, unfolding, internalization and degradation of ubiquitinated protein substrates into peptides by 26S proteasome.

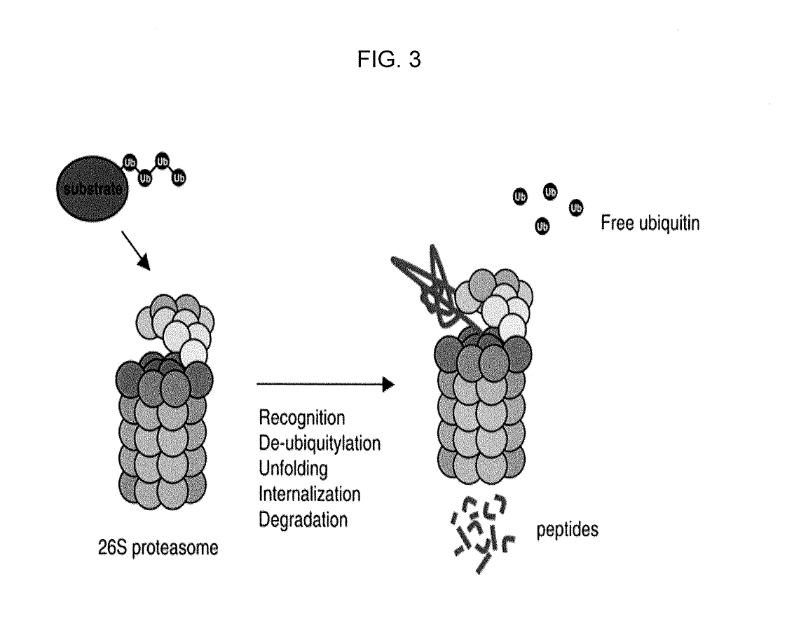

FIG. 4 is a model of structural components of the 26S proteasome in three dimensions with correlations of structure and function.

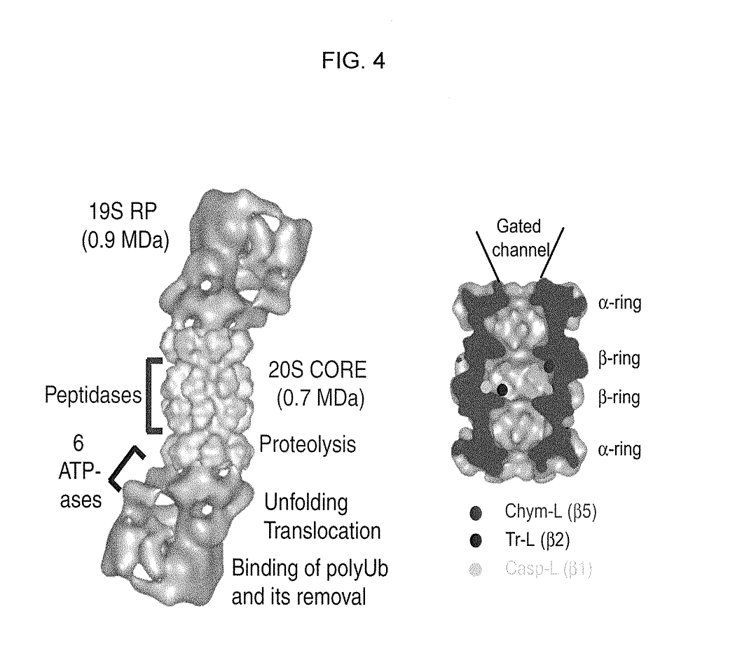

FIG. 5 is a schematic diagram comparing Pup (Prokaryotic Ubiquitin-like protein) proteasome and Ubiquitin Proteasome systems.

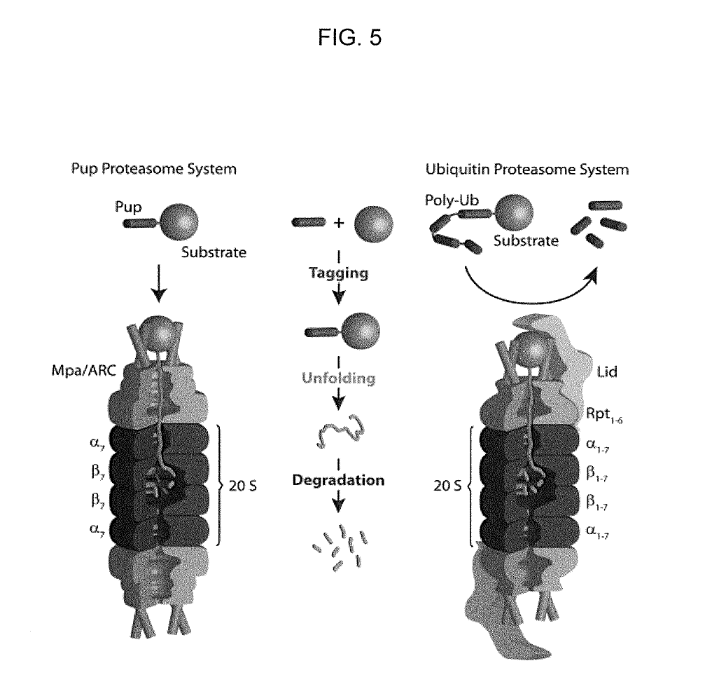

FIG. 6 shows structures of five chemical classes, Aldehydes, Beta-lactones, Boronates, Syringolins and Epoxyketones, for 11 suicide inhibitors of proteasome.

FIG. 7 is a diagram showing substrate mimicry and covalent adduct formation between suicide inhibitors and target enzymes.

FIG. 8 shows details of the chemical structures of Syringolins A, B and E with respect to substituents at R.

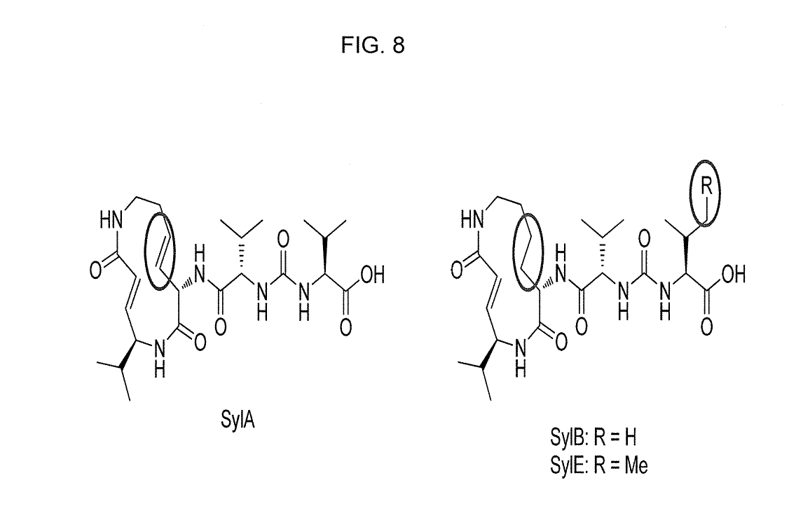

FIG. 9 shows the mechanism of inhibition of proteasomes by Syringolin by forming a covalent adduct.

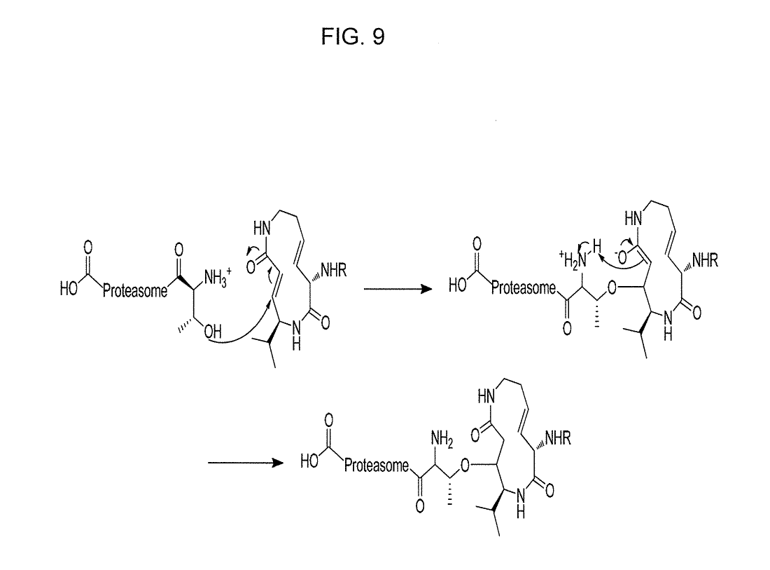

FIG. 10 shows the mechanism for Oxathiazolone—mediated proteasome inhibition.

FIG. 11 shows a chemical structure of an N-acetyl tripeptide aminomethylcoumarin. The M. tuberculosis proteasome differs from the human proteasome in having a strong preference for substrates in which P1 (residue at the scissile bond) is tryptophan, particularly in protein substrates having glycine, proline, lysine or arginine residues are at P3 (two residues of the scissile bond).

FIG. 12 is a schematic drawing of chemical reactions showing syringolin's proteasome inhibition mechanism. Syringolins inhibit proteasomes by conjugate addition to form a proteasome-Syringolin covalent adduct.

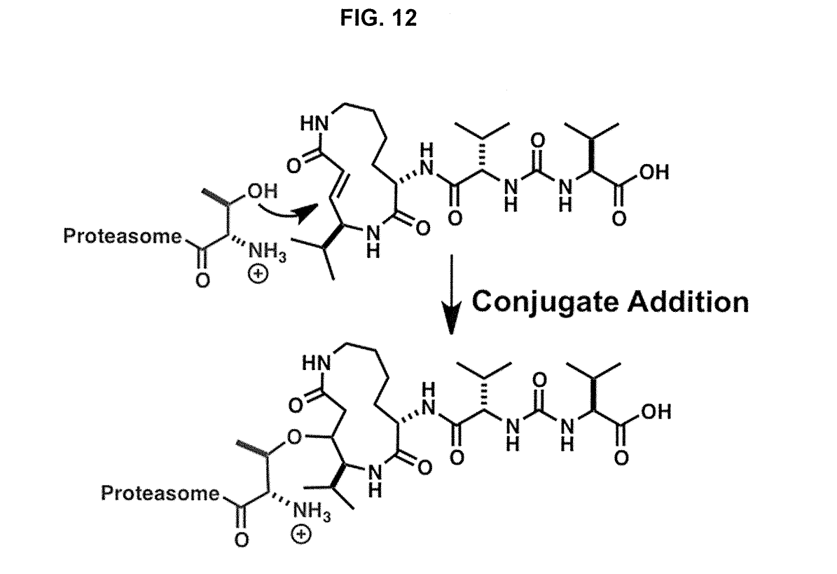

FIG. 13 is a photograph of Streptomyces coelicolor colonies. S. coelicolor is a non-pathogenic bacterial species used as a surrogate for determining the activity of inhibitors of M. tuberculosis proteasome because of high homology between proteasomes of the two bacterial species.

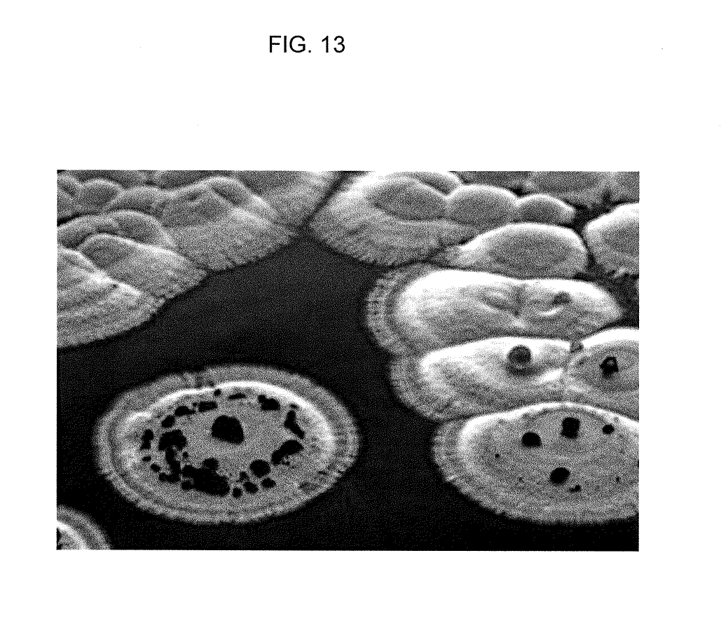

FIG. 14 is a chemical formula for Syringolin B showing the two positions labeled R and R′ that mimic P1 and P3 residue of the enzyme substrate.

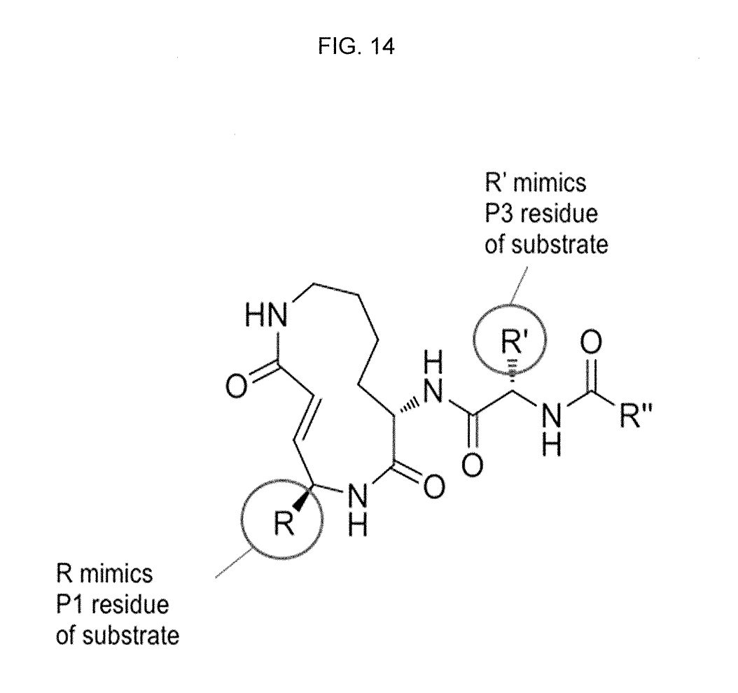

FIG. 15 is a schematic diagram showing retro synthesis of synthetic Syringolins as described in recent publications. Pirrung, M. C., et al. 2012 Org. Lett. 12: 2402-2405.

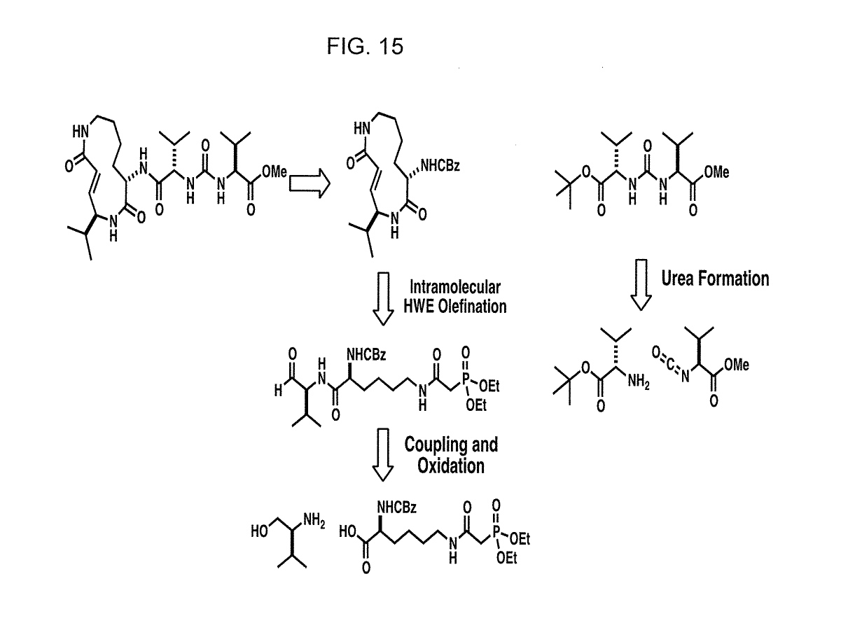

FIG. 16 is a schematic diagram showing a retro synthetic pathway by Modular route of synthetic Syringolins as described. Pirrung, M. C., et al. 2012 Org. Lett. 12: 2402-2405.

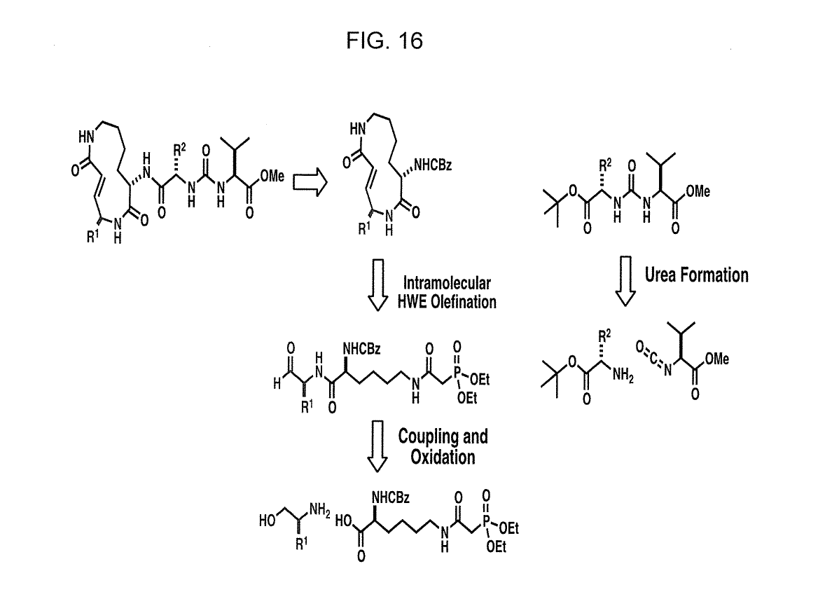

FIG. 17 is a schematic diagram showing retro synthesis by Diversity-oriented route of synthetic Syringolins. The Syringolin B analogs in examples herein were synthesized by diversity-oriented synthesis in which, an amino alcohol is coupled and oxidized to an aldehyde of a substrate such as lysine. The resulting structure is subjected to the Horner-Wadsworth-Emmons (HWE) olefination in which the stabilized phosphonate carbanions react with aldehydes (or ketones) to produce predominantly E-alkenes. The HWE olefination is also a ring closing reaction. The resulting ring reacts with urea to form Syringolin B analogs. Commercially available t-butyl ester amino acids are added to isocyanate to obtain urea.

FIG. 18 is a drawing of a chemical structure of a Syringolin B showing aromatic and aliphatic amino acid substituents of a Syringolin molecule at two positions labeled R¹ and R².

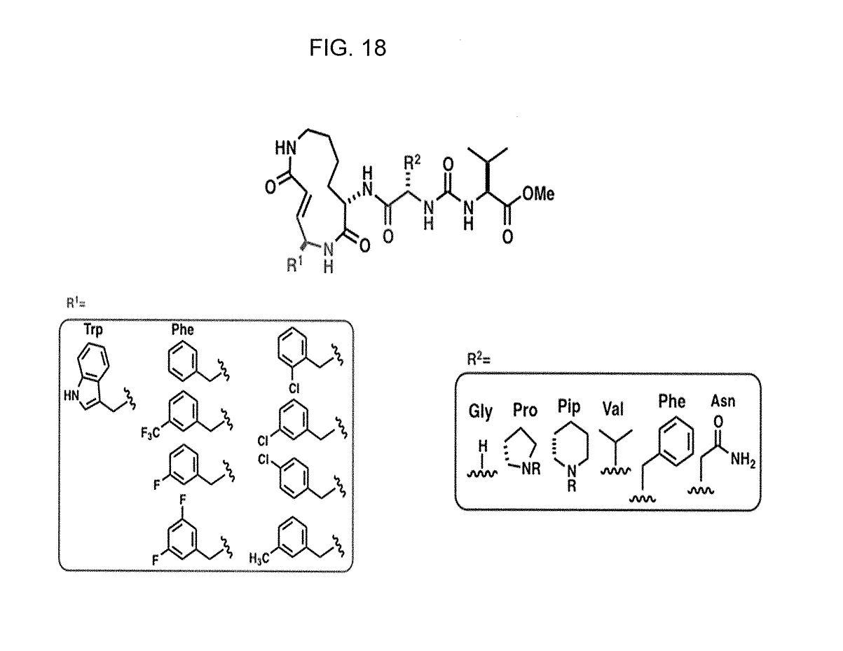

FIG. 19A-FIG. 19M show the structures of Syringolin B and phenyl Syringolin B, the structures of nine phenyl Syringolin B derivatives with substitutions in the phenyl ring.

FIG. 20 shows results of in vivo assay of proteasome activity by aromatic Syringolin B. and the structure of this inhibitor. The assay measures amount of the non-heme chloroperoxidase SCO 0465, and inhibition of proteasome activity by aromatic Syringolin B.

FIG. 21 is a schematic diagram showing enzymatic assay for assessing inhibition of M. tuberculosis proteasome in vitro by using Suc-LLVY-AMC Fluorogenic Substrate.

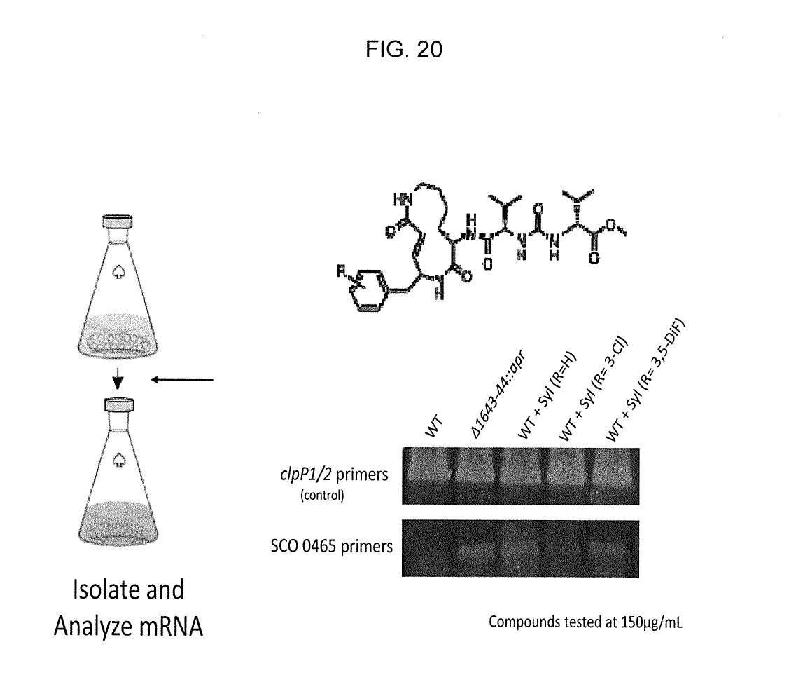

FIG. 22A-FIG. 22C shows chemical structure of Syringolin B and Syringolin B analogs Compound O and Compound P respectively. An overview of proteasome inhibition assay results for Syringolin B, Compound O and Compound P are also shown. The IC₅₀ values for M. tuberculosis and Human proteasome were calculated by performing an enzymatic assay and by plotting peptide hydrolysis rates against the inhibitor concentrations and fitting those data to the Hill form of a dose-response curve.

FIG. 22A shows the ratio of IC₅₀ values for inhibition of M. tuberculosis and Human proteasome by Syringolin B. The data indicate that Syringolin B does not selectively inhibit M. tuberculosis proteasome as compared to human proteasome.

FIG. 22B shows the ratio of IC₅₀ values for inhibition of M. tuberculosis and Human proteasome by Syringolin B analog Compound O. The data indicate that Compound O selectively inhibits M. tuberculosis proteasome as compared to human proteasome. Compound O selectively inhibits M. tuberculosis proteasome more than 1,900 times better than Syringolin B.

FIG. 22C shows the ratio of IC₅₀ values for inhibition of M. tuberculosis and Human proteasome by Syringolin B analog Compound P. The data indicate that Compound P selectively inhibits M. tuberculosis proteasome as compared to human proteasome. Compound P selectively inhibits M. tuberculosis proteasome more than 6,500 times better than Syringolin B and more than three times than Compound O.

FIG. 23 shows Structure Activity Relationship (SAR) analysis of Syringolin B analogs at R position. Four amino acids, tryptophan, phenyalanine, 3-Cl phenylalanine and valine were tested at the R position of Syringolin B. The data show that identity of the residue at R position has little contribution to selectivity for M. tuberculosis proteasome inhibition.

FIG. 24 shows SAR analysis of Syringolin B analogs at R′ position. Five amino acids, glycine, proline, valine, arginine and phenyalanine were tested at the R′ position of Syringolin B. The data show that identity of the residue at R′ position is critical to obtain a compound that selectively inhibits M. tuberculosis proteasome. Glycine at R′ position provided maximum selective inhibition of M. tuberculosis proteasome.

FIG. 25 shows chemical structure of Compound Q, which is an analog of Syringolin P. Compound Q was synthesized by Diversity—oriented synthesis with tryptophan at R position and Phenylalanine at R′ position. The IC₅₀ values of Compound Q for inhibition of M. tuberculosis and Human proteasome were calculated by performing an enzymatic assay and by plotting peptide hydrolysis rates against the inhibitor concentrations and fitting those data to the Hill form of a dose-response curve. The proteasome inhibition assay data show that Compound Q selectively does not inhibit M. tuberculosis proteasome.

FIG. 26A-FIG. 26C show chemical structures and IC₅₀ values calculated by enzymatic assay for Compound P and Compound P analogs Compound P1 and Compound P2 respectively synthesized to test SAR. Compound P is a Syringolin B analog with glycine at position R′ (FIG. 26A). Two variants of Compound P were designed, synthesized and tested. FIG. 26B shows Compound P1 in which the glycine is substituted with a glycine analog having a methyl group replacing Hydrogen. The IC₅₀ values of Compound P1 indicates that this analog does not inhibit M. tuberculosis proteasome or human proteasome. FIG. 26C shows Compound P2 in which glycine is substituted with a glycine analog with additional Carbon atoms. The IC₅₀ values of Compound P2 indicate that this analog also does not inhibit M. tuberculosis proteasome or human proteasome. Therefore, presence of glycine at R′ position is essential for proteasome inhibition and Compound P analogs Compounds P1 and P2 synthesized with glycine analogs do not inhibit bacterial and human proteasomes.

FIG. 27A-FIG. 27C shows results obtained from In Vitro Cell Line Screening Project (IVCLSP) at NIH for bioactivity of Compound P against 60 cancer cell lines. Syringolin B and

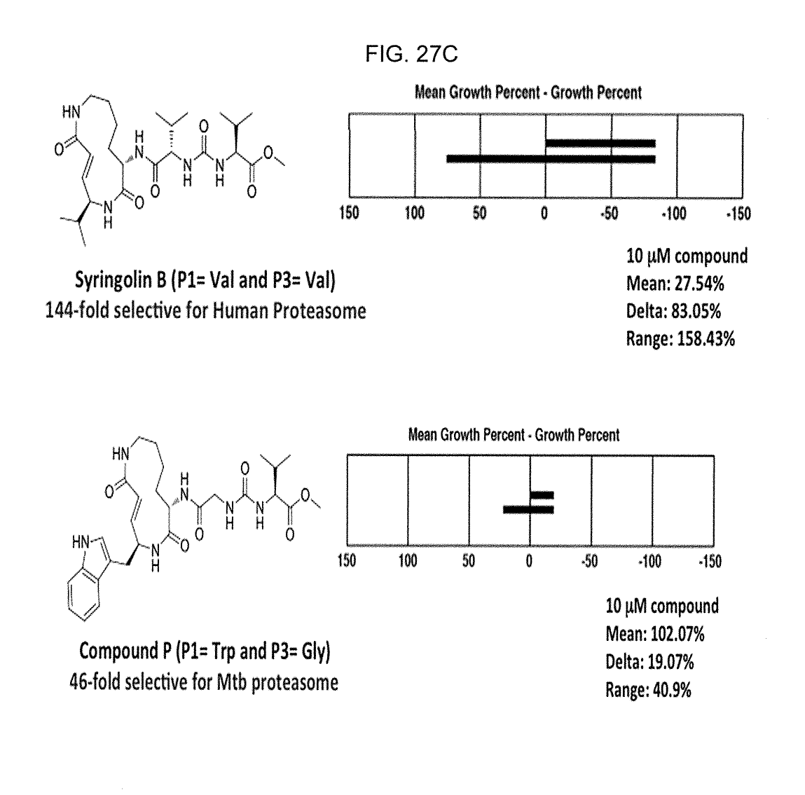

Compound P were tested with NIH's IVCLSP program. The data obtained from the screen are shown in FIG. 27A and FIG. 27B. The data indicate that Compound P does not inhibit cancer cell proteasomes and as a result does not kill cancer cells. Therefore, Compound P inhibition of proteasomes such as M. tuberculosis, S. coelicolor, and M. Bovis BCG is selective for bacterial cells. That Compound P does not inhibit human proteasome indicates that this compound will not be not toxic to human cells. FIG. 27C shows IVCLSP screen data comparison of Syringolin B and Compound P. The data obtained from IVCLSP is consistent with the data obtained from in vitro enzymatic assay. Syringolin B having valine at both R and R′ position is 144 times more selective for human proteasome than M. tuberculosis proteasome and Compound P consisting of tryptophan at R position and Glycine at R′ position is 46 times more selective for M. tuberculosis proteasome than human proteasome.

### DETAILED DESCRIPTION

An increasing number of infections caused by multi-drug resistant bacteria portend a major public health crisis. Boucher H W, et al. 2009 Clinical Infectious Diseases. 48(1):1-12. A contributing factor to the antibacterial drug resistance problem is the fact that most drugs in use today target a relatively small subset of proteins and nucleic acids with essential roles in DNA replication, transcription, translation and cell envelope homeostasis. Walsh, C. 2003 ASM Press, Washington. D.C. USA reviewed by Döhren, H. V. 2004 Protein Sci. 13(11): 3059-3060. Targeting of a small subset of proteins and nucleic acids is problematic because genetic mutations or genes that confer resistance to one antibacterial drug can also confer resistance to others with the same target or mechanism of action. Walsh, C. 2003 ASM Press, Washington, D. C. USA. To circumvent “cross-resistance”, new antibacterial drug targets must be identified and validated. The mycobacterial 20S proteasome is one of the most interesting drug targets. Butler, S. M., et al. 2006 Mol. Microbiol. 60, 553-562; Raju R, et al. 2012 Nature Reviews Drug Discovery.” 11(10):777-89; Roberts, D., M., et al. 2013 Future Microbology. 8, 621-631. Genetic studies of the 20S proteasome indicate that it is essential for the persistence of M. tuberculosis in mice. The 20S proteasome is critical for persistence because it mediates resistance to nitric oxide, a toxic gas produced as part of the innate immune response. Examples herein show that chemical inhibitors of the 20S proteasome support the host immune system clearing M. tuberculosis infections.

Proteasomes are large, multisubunit complexes that catalyze hydrolysis of intracellular proteins. Baumeister, W., et al. 1998 Cell. 92:367-80; Voges, D., et al. 1999 Annu. Rev. Biochem. 68:1015-68. The 26S proteasome contains a 20S proteolytic core and one or two 19S regulatory particles. Ibid. The 20S proteolytic core has a characteristic barrel shaped structure defined by four stacked rings. Each ring contains seven proteins (α and β subunits), each of which has a protease active site oriented toward the interior of the barrel. Ibid. Because the 26S proteasome has a self-compartmentalized structure, substrates of the proteasome must be unfolded before they can be degraded inside the barrel. Substrate identification and unfolding is mediated by the regulatory particles which have ATPases. Hochstrasser, M. 2009 Chem Rev. 109:1479-80; Saeki Y, et al. 2012 Methods Mol. Biol. 2832:315-37.

Proteasomes were considered to be exclusive to eukaryotes and archaea until the discovery of these proteolytic complexes in a Rhodococcus bacterium in 1995. Tamura, T., et al. 1995 Curr Biol. 5:766-74. It is now well known that the actinobacteria (including Mycobacteria, Streptomyces bacteria, Corynebacteria and Frankia) are eubacteria that have proteasomes. Lupas A, Zühl F, et al. 1997 Mol Biol Rep. 24:125-31. The peculiarity of proteasomes in these bacteria has generated much research into their physiological roles. Proteasome null strains of pathogenic M. tuberculosis are hypersensitive to the nitric oxide generated by macrophages. Darwin, K. H., et al. 2003 Science. 302:1963-6. Further, deletion or silencing of the proteasome encoding genes blocks the ability of M. tuberculosis to persist in mice. Gandotra, S., et al. 2007 Nat Med. 13:1515-20; Gandotra, S., et al. 2010 PLoS Pathog. 6:e1001040. From these findings and the fact that persistence is a major complication in the treatment of tuberculosis, the mycobacterial proteasome is a suitable drug target.

Proteasome inhibitors are used for the treatment of cancer, (Kisselev, A. F., et al. 2012 Chem Biol. 19:99-115; Adams, J., et al. 1999 Cancer Res. 59:2615-22; Molineaux, S. M. 2012 Clin. Cancer Res. 18: 15-20). Similarly, it is here envisioned that novel inhibitors of the bacterial proteasome are useful to treat mycobacterial infections such as tuberculosis or leprosy. Butler, S. M., et al. 2006 Mol. Microbiol. 60, 553-562; Raju R, et al. 2012 Nature Reviews Drug Discovery.” 11(10):777-89; Roberts, D., M., et al. 2013 Future Microbology. 8, 621-631. M. tuberculosis null strain are susceptible to nitric oxide similarly small molecule inhibitors of the proteasome sensitize wild-type M. tuberculosis to nitrooxidative stress.

### Small Molecule Inhibitors of the Bacterial Proteasome

Fellutamide B and bortezomib are inhibitors of eukaryotic proteasomes that inhibit the mycobacterial proteasomes. Lin, G., et al. 2010 Arch. Biochem. Biophys. 501: 214-20. Anti-tuberculosis drugs that act by inhibition of the mycobacterial proteasome cross-react with the human proteasome. Cross reaction is barrier for development of anti-tuberculosis drugs. Butler, S. M., et al. 2006 Mol. Microbiol. 60, 553-562, Raju R, et al. 2012 Nature Reviews Drug Discovery.” 11(10):777-89, Roberts, D., M., et al. 2013 Future Microbology. 8, 621-631.

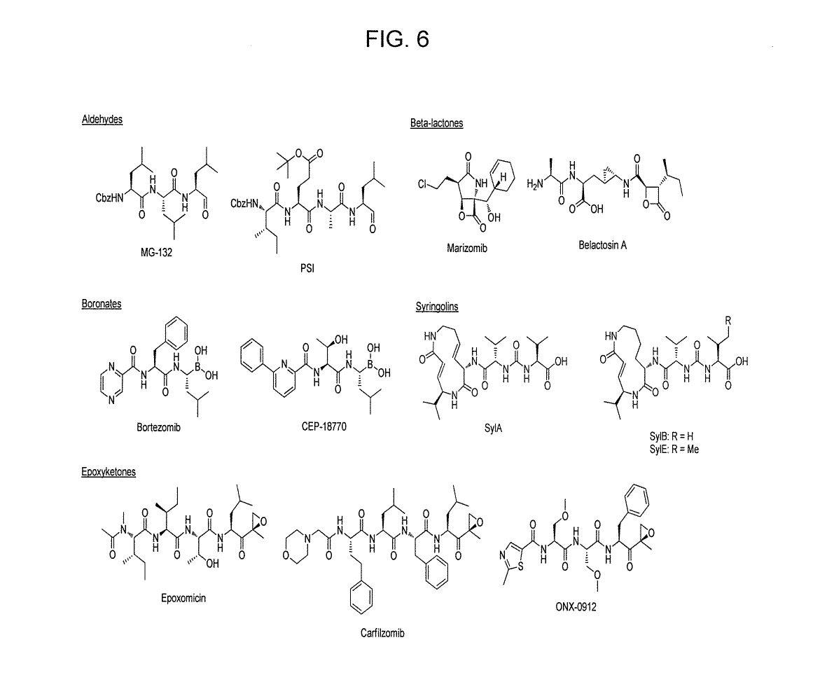

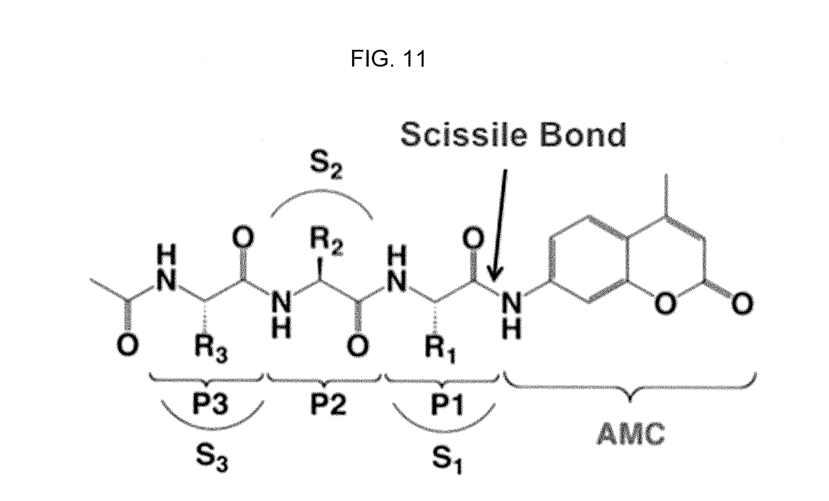

A variety of Bacterial proteasome inhibitors shown in FIG. 6 were characterized including aldehydes such as MG-132 and PSI; Beta-lactones such as Marizomib and Belactosin A; Boronates such as Bortezomib and CEP-18770; Syringolins such as Syringolin A, B and E; and Epoxyketones such as Expoxomicin, Carfilzomib and ONX-0912. To design selective inhibitors, attempts to identify and exploit differences between the substrate specificities of the mycobacterial and human proteasomes have been made. Mycobacterial proteasomes differs from the human proteasome because of a strong preference of the mycobacterial proteasome for substrates in which P1 (residue at the scissile bond) is tryptophan, particularly when glycine, proline, lysine or arginine residues are at P3 (two residues of the scissile bond) as shown in FIG. 11. Lin, G., et al. 2008 J. Biol. Chem. 283:34423-31.

The mycobacterial proteasome substrate preferences were exploited herein in the design of selective inhibitors of the mycobacterial proteasome. Specifically, a bortezomib analog in which the leucine mimic at P1 was replaced with a tryptophan mimic (i.e., meta-chlorophenyl) exhibited an 8,000-fold improvement in potency and 8-fold enhanced species selectivity. Ibid. A library of 16,000 “N,C-capped dipeptides” (many of which had non-proteinogenic amino acid constituents) with structures reminiscent of mycobacterial proteasome substrates has been screened and low nanomolar inhibitors that were up to about 1,500-fold more selective for the bacterial proteasome were identified. Lin, G., et al. 2013 J. Am. Chem. Soc., 135, 9968-9971. The best compounds obtained were observed to have sensitized wild-type M. tuberculosis to nitro-oxidative stress at concentrations between about 1-25 micromolar. However, the toxicity of these selected mycobacterial proteasome substrates to human cells or their efficacies in models of tuberculosis infection has not been analyzed. Examples herein show that substrate mimicry is an effective strategy for the design of selective inhibitors of the mycobacterial enzyme.

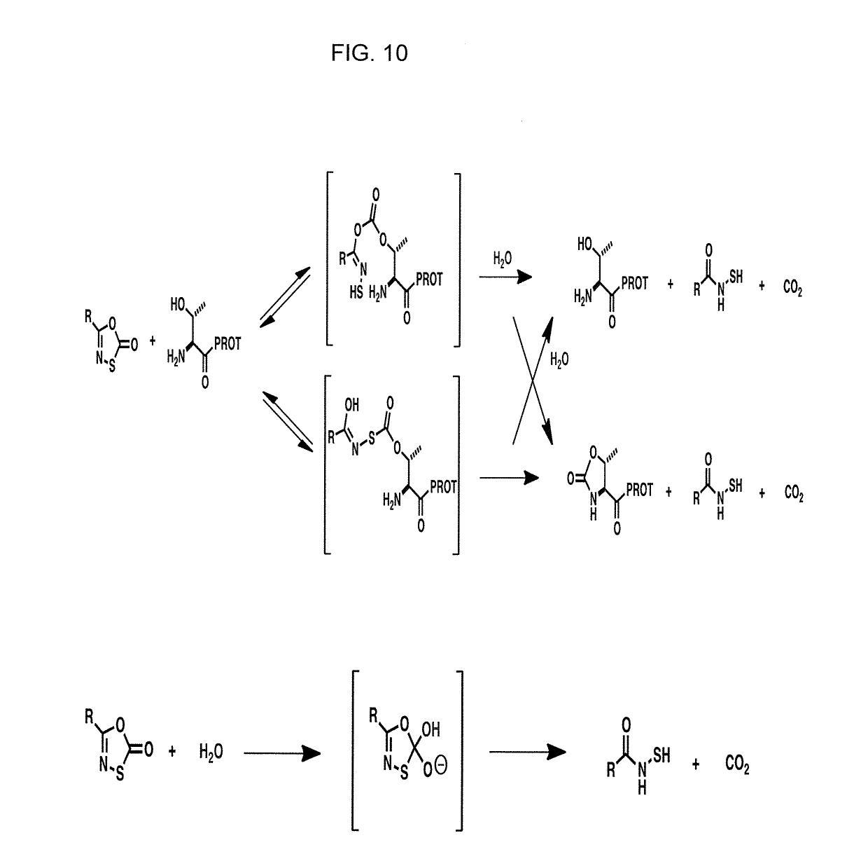

The most selective inhibitors of the mycobacterial proteasome are the oxathiazol-2-ones. Lin, G., et al. 2009 Nature. 461: 621-6. The oxathiazol-2-ones molecules irreversibly inhibit the mycobacterial proteasome and have about 1,300-fold selectivity for the mycobacterial proteasome compared to the human proteasome. The mechanism for oxathiazol-2-ones mediated proteasome inhibition is shown in FIG. 10. The oxathiazol-2-ones react with the active site threonine residue in a cyclocarbonylation, yielding a carbamate. Ibid. The covalently modified proteasome is catalytically inactive. Even though oxathiazol-2-ones are reported to render M. tuberculosis susceptible to nitrooxidative stress in vitro, it is unlikely that these compounds will be useful in the treatment of tuberculosis because of chemical instability (i.e., fast rates of hydrolysis) in serum. Additionally, hydrolysis of the inhibitor enzyme intermediate allows the proteasome to degrade oxathiazol-2-ones without losing activity.

No new structural classes of antibacterial drugs were introduced between 1962 and 2000, indicating a troubling innovation gap in infectious disease medicine. Walsh, C. T., et al. 2014 J. Antibiotics, 67, 7-22. The examples herein fill this gap by developing antibacterial agents that target proteolytic complexes. Any drug candidates emerging from a discovery program developed specifically to inhibit these enzymes would be of great value because the candidates would be free of the cross-resistance that otherwise adversely affects drug development into the clinic. In addition to their potential as transformative bactericidal drugs, small molecule modulators of bacterial proteolytic complexes for the suppression of virulence in pathogenic bacteria represent a new strategy for antibacterial therapy.

### Pharmaceutical Compositions

An aspect of the present invention provides pharmaceutical compositions that are Syringolin analogs. In general embodiments, the pharmaceutical composition is compounded as an oral formulation for administration to a subject. In related embodiments, the pharmaceutical composition is formulated sufficiently pure for administration to a human subject, e.g., oral or systemic delivery route into a human subject. In certain embodiments, these compositions optionally further include one or more additional therapeutic agents. In certain embodiments, the additional therapeutic agent or agents are selected from the group consisting of growth factors, anti-inflammatory agents, vasopressor agents including but not limited to nitric oxide and calcium channel blockers, collagenase inhibitors, topical steroids, matrix metalloproteinase inhibitors, ascorbates, angiotensin II, angiotensin III, calreticulin, tetracyclines, fibronectin, collagen, thrombospondin, transforming growth factors (TGF), keratinocyte growth factor (KGF), fibroblast growth factor (FGF), insulin-like growth factors (IGFs), IGF binding proteins (IGFBPs), epidermal growth factor (EGF), platelet derived growth factor (PDGF), neu differentiation factor (NDF), hepatocyte growth factor (HGF), vascular endothelial growth factor (VEGF), heparin-binding EGF (HBEGF), thrombospondins, von Willebrand Factor-C, heparin and heparin sulfates, and hyaluronic acid.

In other embodiments, the additional agent is a compound, composition, biological or the like that potentiates, stabilizes or synergizes or even substitutes for the ability of Syringolin B analogs. Also included are therapeutic agents that may beneficially or conveniently be provided at the same time as the Syringolin B analogs, such as agents used to treat the same, a concurrent or a related symptom, condition or disease. In some embodiments, the drug may include without limitation an anti-tumor, antiviral, antibacterial, anti-mycobacterial, anti-fungal, anti-proliferative or anti-apoptotic agent. Drugs that are included in the compositions of the invention are well known in the art. See for example, Goodman & Gilman's The Pharmacological Basis of Therapeutics, 9th Ed., Hardman, et al., eds., McGraw-Hill, 1996, the contents of which are herein incorporated by reference herein.

As used herein, the term “pharmaceutically acceptable carrier” includes any and all solvents, diluents, or other liquid vehicle, dispersion or suspension aids, surface active agents, isotonic agents, thickening or emulsifying agents, preservatives, solid binders, lubricants and the like, as suited to the particular dosage form desired. Remington's Pharmaceutical Sciences Ed. by Gennaro, Mack Publishing, Easton, Pa., 1995 provides various carriers used in formulating pharmaceutical compositions and known techniques for the preparation thereof. Some examples of materials which can serve as pharmaceutically acceptable carriers include, but are not limited to, sugars such as glucose and sucrose; excipients such as cocoa butter and suppository waxes; oils such as peanut oil, cottonseed oil, safflower oil, sesame oil, olive oil, corn oil, and soybean oil; glycols such a propylene glycol; esters such as ethyl oleate and ethyl laurate; agar; buffering agents such as magnesium hydroxide and aluminum hydroxide; alginic acid; pyrogen-free water; isotonic saline; Ringer's solution; ethyl alcohol; and phosphate buffer solutions, as well as other non-toxic compatible lubricants such as sodium lauryl sulfate and magnesium stearate, as well as coloring agents, releasing agents, coating agents, preservatives and antioxidants can also be present in the composition, according to the judgment of the formulator.

### Therapeutically Effective Dose

Treatment of a bacterial infection by compositions and methods provided herein involves contacting a tissue or cells with a pharmaceutical composition, for example, administering a therapeutically effective amount of a pharmaceutical composition having as an active agents Syringolin B analog, to a subject in need thereof, in such amounts and for such time as is necessary to achieve the desired result. Methods for example include treating M. tuberculosis infection by treating with Syringolin B analog.

The compositions, according to the method of the present invention, may be administered using any amount and any route of administration effective for treating M. tuberculosis or other complement-related diseases and conditions. Thus, the expression “amount effective for treating M. tuberculosis”, as used herein, refers to a sufficient amount of composition to beneficially prevent or ameliorate the symptoms of M. tuberculosis.

The exact dosage is chosen by the individual physician in view of the patient to be treated. Dosage and administration are adjusted to provide sufficient levels of the active agent(s) or to maintain the desired effect. Additional factors which may be taken into account include the severity of the disease state, e.g., intermediate or advanced stage of M. tuberculosis or other bacterial infections; age, weight and gender of the patient; diet, time and frequency of administration; route of administration; drug combinations; reaction sensitivities; and tolerance/response to therapy. Long acting pharmaceutical compositions might be administered hourly, twice hourly, every 3 to four hours, daily, twice daily, every 3 to 4 days, every week, or once every two weeks depending on half-life and clearance rate of the particular composition.

The active agents of the invention are preferably formulated in dosage unit form for ease of administration and uniformity of dosage. The expression “dosage unit form” as used herein refers to a physically discrete unit of active agent appropriate for the patient to be treated. It will be understood, however, that the total daily usage of the compositions of the present invention will be decided by the attending physician within the scope of sound medical judgment. For any active agent, the therapeutically effective dose can be estimated initially either in cell culture assays or in animal models, as provided herein, usually mice, but also potentially from rats, rabbits, dogs, or pigs. The animal cell model provided herein is also used to achieve a desirable concentration and total dosing range and route of administration. Such information can then be used to determine useful doses and routes for administration in humans.

A therapeutically effective dose refers to that amount of active agent that ameliorates the symptoms or condition of M. tuberculosis. Therapeutic efficacy and toxicity of active agents can be determined by standard pharmaceutical procedures in cell cultures or experimental animals, e.g., ED50 (the dose is therapeutically effective in 50% of the population) and LD50 (the dose is lethal to 50% of the population). The dose ratio of toxic to therapeutic effects is the therapeutic index, and it can be expressed as the ratio, LD50/ED50. Pharmaceutical compositions which exhibit large therapeutic indices are preferred. The data obtained from cell culture assays and animal studies are used in formulating a range of dosage for human use.

The daily dosage of the products may be varied over a wide range, such as from 0.001 to 800 mg per adult human per day. For ocular administration, the compositions are preferably provided in the form of a pill or a solution containing 0.001, 0.01, 0.05, 0.1, 0.5, 1.0, 2.5, 5.0, 10.0, 15.0, 25.0, 50.0, 100.0, 250.0, or 500.0 micrograms of the active ingredient for the symptomatic adjustment of the dosage to the patient to be treated.

A unit dose typically contains from about 0.001 milligrams to about 500 milligrams of the active ingredient, preferably from about 0.1 milligrams to about 100 milligrams of active ingredient, more preferably from about 1.0 milligrams to about 10 milligrams of active ingredient. An effective amount of the drug is ordinarily supplied at a dosage level of from about 0.0001 mg/kg to about 25 mg/kg of body weight per day. For example, the range is from about 0.001 to 10 mg/kg of body weight per day, or from about 0.001 mg/kg to 1 mg/kg of body weight per day. The compositions may be administered on a regimen of, for example, one to four or more times per day. A unit dose may be divided for example, administered in two or more divided doses.

### Administration of Pharmaceutical Compositions

As formulated with an appropriate pharmaceutically acceptable carrier in a desired dosage, the pharmaceutical composition provided herein is administered to humans and other mammals topically such as oral (as by solutions, ointments, or drops), nasally, bucally, orally, rectally, parenterally, intracisternally, intravaginally, or intraperitoneally.

Liquid dosage forms for oral, or other systemic administration include, but are not limited to, pharmaceutically acceptable emulsions, microemulsions, solutions, suspensions, syrups and elixirs. In addition to the active agent(s), the liquid dosage forms may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the ocular, oral, or other systemically-delivered compositions can also include adjuvants such as wetting agents, and emulsifying and suspending agents.

Dosage forms for topical or transdermal administration of an inventive pharmaceutical composition include ointments, pastes, creams, lotions, gels, powders, solutions, sprays, inhalants, or patches, for example for treatment of other mycobacterial species such as M. leprae. The active agent is admixed under sterile conditions with a pharmaceutically acceptable carrier and any needed preservatives or buffers as may be required. For example, ocular or cutaneous routes of administration are achieved with aqueous drops, a mist, an emulsion, or a cream. Administration may be therapeutic or it may be prophylactic. Devices may be coated with, impregnated with, bonded to or otherwise treated with a composition as described herein.

Transdermal patches have the added advantage of providing controlled delivery of the active ingredients to the body. Such dosage forms can be made by dissolving or dispensing the compound in the proper medium. Absorption enhancers can also be used to increase the flux of the compound across the skin. The rate can be controlled by either providing a rate controlling membrane or by dispersing the compound in a polymer matrix or gel.

Injectable preparations, for example, sterile injectable aqueous or oleaginous suspensions may be formulated according to the known art using suitable dispersing or wetting agents and suspending agents. The sterile injectable preparation may also be a sterile injectable solution, suspension or emulsion in a nontoxic parenterally acceptable diluent or solvent, for example, as a solution in 1,3-butanediol. Among the acceptable vehicles and solvents that may be employed are water, Ringer's solution, U.S.P. and isotonic sodium chloride solution. In addition, sterile, fixed oils are conventionally employed as a solvent or suspending medium. For this purpose any bland fixed oil can be employed including synthetic mono- or diglycerides. In addition, fatty acids such as oleic acid are used in the preparation of injectables. The injectable formulations can be sterilized, for example, by filtration through a bacterial-retaining filter, or by incorporating sterilizing agents in the form of sterile solid compositions which can be dissolved or dispersed in sterile water or other sterile injectable medium prior to use. In order to prolong the effect of an active agent, it is often desirable to slow the absorption of the agent from subcutaneous or intramuscular injection. Delayed absorption of a parenterally administered active agent may be accomplished by dissolving or suspending the agent in an oil vehicle. Injectable depot forms are made by forming microencapsule matrices of the agent in biodegradable polymers such as polylactide-polyglycolide. Depending upon the ratio of active agent to polymer and the nature of the particular polymer employed, the rate of active agent release can be controlled. Examples of other biodegradable polymers include poly(orthoesters) and poly(anhydrides). Depot injectable formulations are also prepared by entrapping the agent in liposomes or microemulsions which are compatible with body tissues.

Compositions for rectal or vaginal administration are preferably suppositories which can be prepared by mixing the active agent(s) of this invention with suitable non-irritating excipients or carriers such as cocoa butter, polyethylene glycol or a suppository wax which are solid at ambient temperature but liquid at body temperature and therefore melt in the rectum or vaginal cavity and release the active agent(s).

Solid dosage forms for oral administration include capsules, tablets, pills, powders, and granules. In such solid dosage forms, the active agent is mixed with at least one inert, pharmaceutically acceptable excipient or carrier such as sodium citrate or dicalcium phosphate and/or a) fillers or extenders such as starches, sucrose, glucose, mannitol, and silicic acid, b) binders such as, for example, carboxymethylcellulose, alginates, gelatin, polyvinylpyrrolidinone, sucrose, and acacia, c) humectants such as glycerol, d) disintegrating agents such as agar-agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof.

Solid compositions of a similar type may also be employed as fillers in soft and hard-filled gelatin capsules using such excipients as milk sugar as well as high molecular weight polyethylene glycols and the like. The solid dosage forms of tablets, dragees, capsules, pills, and granules can be prepared with coatings and shells such as enteric coatings, release controlling coatings and other coatings well known in the pharmaceutical formulating art. In such solid dosage forms the active agent(s) may be admixed with at least one inert diluent such as sucrose or starch. Such dosage forms may also comprise, as is normal practice, additional substances other than inert diluents, e.g., tableting lubricants and other tableting aids such a magnesium stearate and microcrystalline cellulose. In the case of capsules, tablets and pills, the dosage forms may also comprise buffering agents. They may optionally contain opacifying agents and can also be of a composition that they release the active agent(s) only, or preferentially, in a certain part of the intestinal tract, optionally, in a delayed manner. Examples of embedding compositions which can be used include polymeric substances and waxes.

Incorporated by reference in its entirety is the journal article: “Study of PcaV from Streptomyces coelicolor yields new insights into ligand-responsive MarR family of transcription factors”, Nucleic Acids Research, Feb. 8, 2013, pages 1-13, authors Jennifer R. Davis, Breann L. Brown, Rebecca Page, and Jason K. Sello. This paper describes a transcriptional regulator of the bacterial species Streptomyces coelicolor, used in embodiments of the claims for assaying inhibition of proteasome activity by compositions herein. Also incorporated by reference in its entirety is the journal article “Mining the antibiotic resistome”, Chemistry and Biology, 2012, vol. 19, 1220-1221, authored by Jason K Sello, which describes the reservoir of drug resistance genes, the resistome, encoded by microorganisms, and the importance of understanding the function of the resistome for producing novel anti-bacterial compositions effective against multi drug resistant pathogens.

The invention having now been fully described, it is further illustrated by the following examples and claims, which are illustrative and are not meant to be further limiting.

The invention now having been fully described, it is further exemplified by the following examples and claims. Those skilled in the art will recognize or be able to ascertain using no more than routine experimentation, numerous equivalents to the specific procedures described herein. Such equivalents are within the scope of the present invention and claims. The contents of all references including issued patents and published patent applications cited in this application are hereby incorporated by reference in their entirety.

### EXAMPLES

### Example 1

### A Cell-Based Assay to Discover and Evaluate Inhibitors of the Actinobacterial Proteasome

To rapidly discover and evaluate small molecule inhibitors of the mycobacterial proteasome, a cell-based assay that is compatible with high-throughput screening was developed. The pathogenicity of M. tuberculosis and the fact it is susceptible to proteasome inhibitors only in the presence of nitric oxide complicates assays in this organism. Lin, G., et al. 20081 Biol. Chem. 283:34423-31; Lin, G., et al. 2013 J. Am. Chem. Soc., 135, 9968-9971; Lin, G., et al. 2009 Nature. 461: 621-6. Therefore, non-pathogenic actinobacterial species Streptomyces coelicolor was chosen for this example. The proteasome in S. coelicolor is not essential for viability in contrast to M. tuberculosis proteasome which is essential for viability. Nagy, I., et al. 1998 J. Bacteriol. 180:5448-53. A proteasome null strain of S. coelicolor up-regulates production of a haloperoxidase. De Mot, R., et al. 2007 Arch. Microbiol. 188: 257-271. RT-PCR data shows that transcription of the peroxidase gene is significantly up-regulated in a proteasome null strain of S. coelicolor or if the wild-type strain is treated with a proteasome inhibitor (i.e., bortezomib). Assessment of the peroxidase gene transcription is an effective assay for perturbation of the proteasome, however this assay is too cumbersome for high-throughput assays. Therefore, peroxidase gene transcription assay was successfully used to establish the activity of a rationally designed proteasome inhibitor as shown in examples herein.

### Example 2

### In Vivo Assay of Proteasome Inhibition

Syringolin B was selected as a potential proteasome targeted small molecular weight inhibitor, and was tested for proteasome inhibition activity in S. coelicolor using a non-heme chloroperoxidase SCO0456 gene based assay. A mutant S. coelicolor bacterial strain with a deletion of a gene encoding a portion of the 20s proteasome was observed to up regulate a gene SCO0456 which encodes a non-heme chloroperoxidase. De Mot, R, et al. 2007 Archives of Microbiology, 188(3), 257-271. The S. coelicolor mutant lacking 20s proteasome was constructed by procedure as described in De Mot, R, et al. 2007. Three Syringolin B analogs with substituent organic groups at the R position were tested. The three Syringolin B analogs that differ at the R position are substituted, respectively with an hydrogen atom, a 3-Cl group, and 3,5-DiF group respectively. The three Syringolin B analogs were tested for proteasome inhibition in S. coelicolor cultures.

S. coelicolor A3(2) wild type and mutant strains were grown in CYG medium without NaCl (Casitone peptone, 10 g/l; yeast extract, 5 g/l; glucose, 5 g/l) at 30° C. Antibiotics were added as follows: thiostrepton, 10 μg/ml (in liquid medium) or 50 μg/ml (in solid medium); kanamycin, 50 μg/ml. The CYG medium was supplemented with 5 mM MgCl2 and 0.5% (w/v) glycine to grow cells for the preparation of protoplasts. To generate spores, Streptomyces cells were sprayed on MS plates (soya Xour 20 g/l, mannitol 20 g/l, agar 20 g/l, tap water) and incubated for 7 days at 30° C. S. coelicolor cultures for proteome analysis were grown aerobically in tryptic soy broth (TSB).

Spores of S. coelicolor wild-type and mutant strains were used to inoculate l-l baffle flasks containing 250 ml of TSB medium at a density of 5×10⁴ spores per ml. Cells were grown aerobically for 36 h at 30° C. and recovered by centrifugation. Growth curve analysis based on determination of mycelial mass showed that wild-type and mutant cells thus recovered were at late exponential phase, reaching a cell density (dry weight) of 4 mg/ml.

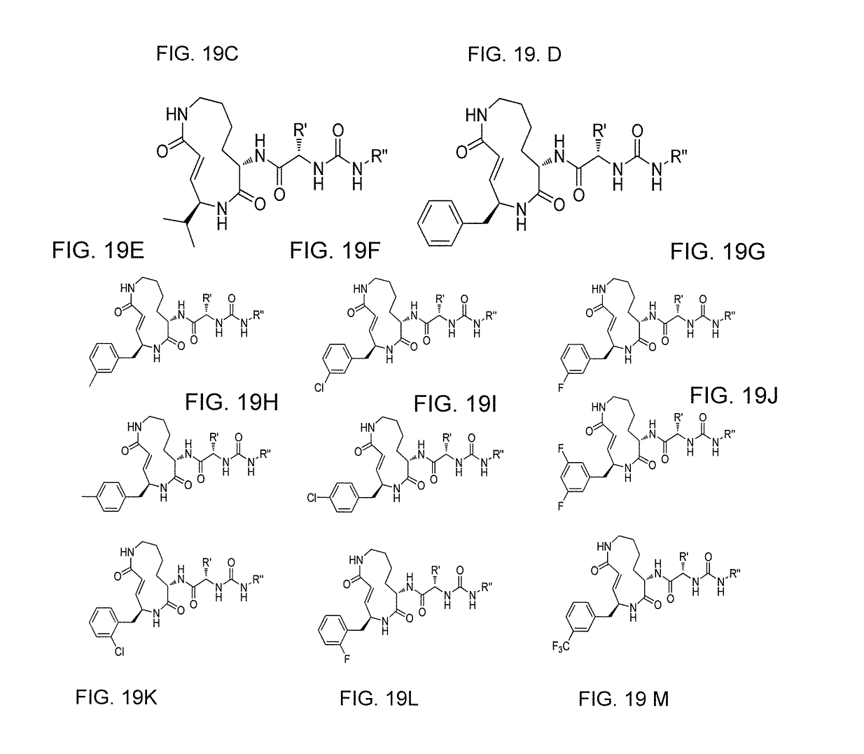

The three Syringolin analogs at a concentration of 150 μg/ml were added to separate wild type flasks and incubated at 30° C. for 5 hours. A 5 ml culture sample was pelleted and RNA was extracted from the mycelium pellet using a standard RNA prep kit. The RNA samples were analyzed by gel electrophoreses using clpP1/2 (control) and SCO0456 primers. The data obtained is shown in FIG. 20.

The gel electrophoresis data show that SCO0456 is up regulated in the mutant S. coelicolor sample and wild-type S. coelicolor samples treated with Syringolin B. Therefore, Syringolin B analogs are likely to be inhibitors of the bacterial proteasome.

### Example 3

### Screening Libraries of Compounds for Modulators of Function of the Bacterial Proteasome

For cell-based assays, plasmids in which the promoter of the peroxidase gene was fused to the neo or the lux reporter gene were introduced into S. coelicolor. The readouts in the assays were either growth of cells in kanamycin supplemented media or bioluminescence.

Compounds were screened individually or in pools of 10-20 compounds in compound library at New England Regional Centers of Excellence in Biodefense at Harvard Medical School and the compound library at the Broad Institute. Molecules identified in the screens were validated in the secondary in vitro enzymatic assays described herein.

The screen identifies modulators of the proteasome. As a complement to secondary enzymatic assays a tertiary assay was conducted that utilizes a cell-based Western blot using Pup-specific antibodies. Burns, K. E., et al. 2012 Methods Mol. Biol. 832:151-60. The tertiary assay was based on the fact that pupylated proteins accumulate in cells in which the proteasome has been inhibited. Small molecule screens using in vitro enzymatic assays were performed. Additionally, efficacies of compound “hits” were tested in nitrooxidative stress assays with cells of each of Bacillus Calmette-Guérin (BCG) and M. tuberculosis.

### Example 4

### Structure-Guided Optimization of a Proteasome Inhibitor

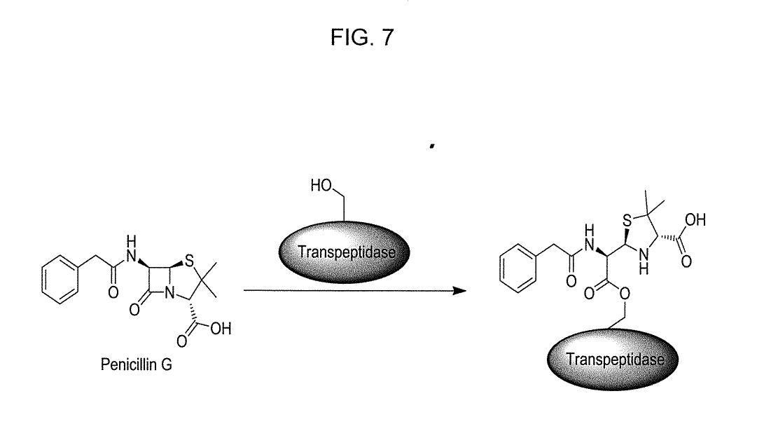

The structures of the syringolins, natural products that inhibit the eukaryotic proteasome, (Krahn, D., et al. 2011 Nat. Prod Rep. 28, 1854-1867) have been optimized for selective inhibition of the bacterial proteasome. The syringolins were chosen as a starting point for optimization because they are reported to have a high degree of selectivity for the chymotrypsin-like activity of the eukaryotic proteasome. Ibid. This selectivity is of particular importance because the bacterial proteasome has only chymotrypsin-like activity. The syringolins are suicide inhibitors of the proteasome, possessing an α,β-unsaturated amide moiety with which the active site threonine of the proteasome subunits react. Ibid., Kisselev, A. F., et al. 2012 Chem Biol. 19:99-115. Syringolins inhibit proteasomes by conjugate addition to form proteasome-Syringolin covalent adduct as shown in FIG. 7.

Based on crystallographic evidence, it is here envisioned that syringolin molecules mimic a peptide substrate of the proteasome. The substituent on the syringolin macrocycle mimics the amino acid residue at the substrate's P1 site (residue of the scissile bond), whereas the peptidyl-side chain mimics the amino acid residue at the substrate's P3 site (two residues upstream of the scissile bond). Since the mycobacterial proteasome has a strong preference for substrates in which P1 is tryptophan in an amino acid sequence in which either glycine, proline, lysine or arginine residues are at P3, analogs of syringolin B were prepared in which the isopropyl side chain of the macrocyle that mimics a valine at P1 was replaced by an indole reminiscent of tryptophan. In one compound, the valine residue (mimicking a residue in the substrate P3 site) in the peptidyl side chain was replaced with proline. In another compound, the valine residue (mimicking a residue in the substrate P3 site) in the peptidyl side chain was replaced with glycine. These novel compounds were prepared by a synthetic route described in Pirrung, M. C., et al. 2012 Org. Lett. 12: 2402-2405.

Assays with mycobacterial enzyme and with human enzyme were used to assess potency and selectivity of the rationally designed syringolin B analogs. The data show that the analog with the methyl indole on the macrocycle and the proline residue in the side chain (compound O) has an IC50 of 2.67 micromolar and that it is 13.4-fold more selective for the mycobacterial proteasome compared to the human enzyme. The analog with the methyl indole on the macrocycle and the glycine residue in the side chain (Compound P) has an IC₅₀ of 0.114 nanomolar and which is 45.6-fold more selective for mycobacterial proteasome than for the human enzyme.

Syringolin B and the analog, Compound P were also tested for their capacity to induce the transcription of the proteasome-dependent peroxidase gene in S. coelicolor, as described in examples herein. The data obtained from this assay show that in comparison to the natural product the rationally designed Syringolin B analog induced transcription of the peroxidase reporter gene.

### Example 5

### Rational Optimization of Syringolins for Inhibition of the Mycobacterial Proteasome

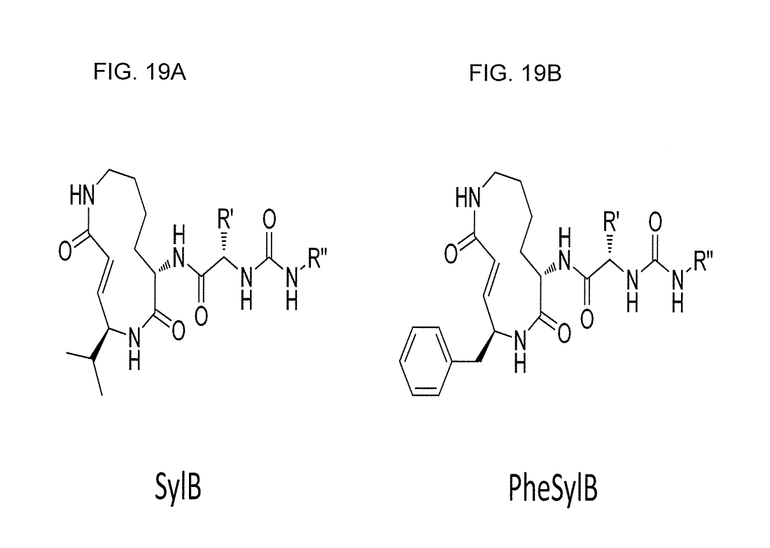

A more extensive set of Syringolin B analogs were prepared by using the strategy described in examples herein. The compounds were synthesized to have tryptophan mimics (indole or aromatic moieties) appended to the macrocycle with peptidyl side chains containing various proteinogenic and non-proteinogenic amino acids (R and R′) mimicking proteasome substrates as shown in FIG. 19A-FIG. 19M. At least 25 different compounds are prepared using a synthetic technique described in Pirrung, M. C., et al. 2012 Org. Lett. 12: 2402-2405. The efficacies of the compounds in cells were assessed in a reporter strain or in peroxidase gene transcription assays. In parallel experiments, potencies and selectivities were measured in in vitro assays with both the mycobacterial and human proteasomes.

### Example 6

### Syringolin B Analog Prepared by Diversity-Oriented Synthesis

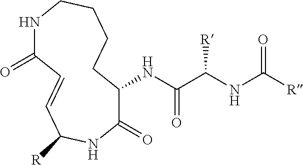

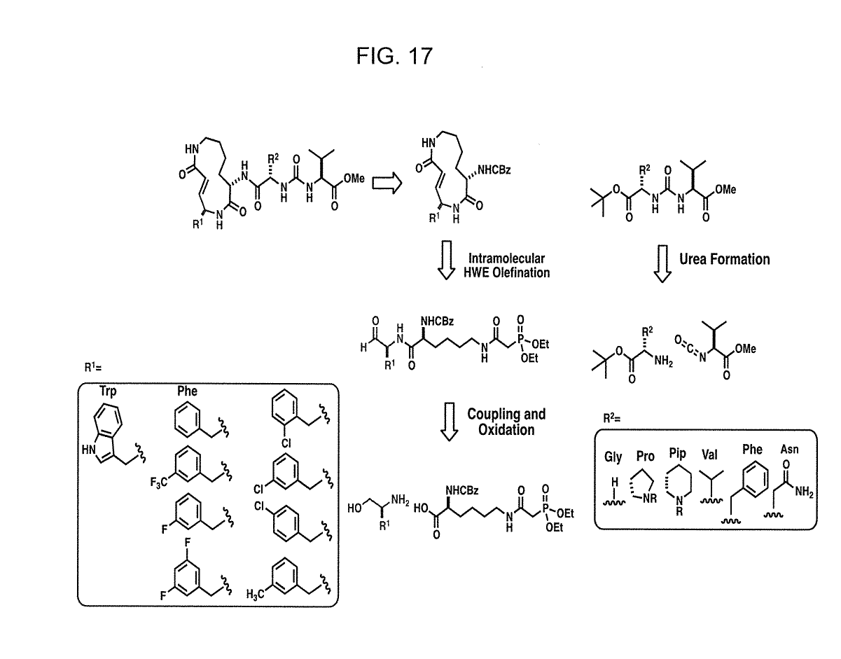

Proteasome inhibitor compound Syringolin B analogs were designed and synthesized by diversity oriented synthesis protocol as shown in FIG. 17. In diversity-oriented synthesis, an amino alcohol is coupled and oxidized to an aldehyde of a substrate such as lysine. The resulting structure is subjected to the Horner-Wadsworth-Emmons (HWE) olefination in which the stabilized phosphonate carbanions react with aldehydes (or ketones) to produce predominantly E-alkenes. The HWE olefination is also a ring closing reaction. The resulting ring reacts with urea to form Syringolin B analogs. Commercially available t-butyl ester amino acids are added to isocynate to obtain urea.

More than 30 syringolin analogs were synthesized and evaluated. Two analogs Compound O and Compound P were found to be promising. The two compounds differ at groups at positions R and R′. Compound O contains a Tryptophan at position R and a Proline at position R′. Compound P contains a Tryptophan at position R and a Glycine at position R′.

### Example 7

### Enzymatic Assay for Assessing Inhibition of M. tuberculosis Proteasome in Vitro

Assays were developed to rapidly assess proteasome activity from M. tuberculosis in a 96- or 384-well multi-well plate format. The Mycobacterium tuberculosis 20S peptidase (mtb 20S) was expressed according to established protocols. Okandeji, B. O., et al. 2009 Journal of Organic Chemistry. 74: 5067-5070; Barthelme, D., et al. 2012 Science 337, 843-6. Peptide hydrolysis by mtb20S was assessed in the presence of Cdc48^{ΔN}, an archaeal/eukaryotic proteasome activator. Barthelme, D., et al. 2012 Science 337, 843-6; Barthelme, D., et al. 2013 Proc Natl Acad Sci USA 110, 3327-32. Peptidolysis was measured in the presence of amounts of 20S inhibitors. The inhibitors were pre-incubated with enzyme before addition of the Suc-LLVY-Amc fluorogenic substrate. Peptide cleavage was then followed continuously with a spectrofluorimeter.

The compounds O and P were evaluated for proteasome inhibition activity using the β5-selective fluorogenic substrate succinyl-leucine-leucine-valine-tyrosine-4-methyl-7-courmarylamide (Suc-LLVY-AMC). Cells were cultured in appropriate media and cell extracts were prepared. The cell extracts were diluted to 200 μg/mL in 5 mmol/L EDTA (pH8.0) and dispensed into a 96-well black opaque plate to give 10 μg protein per reaction. Reactions were initiated by addition of 150 μL of 20 mmol/L HEPES (pH 7.4), containing 0.5 mmol/L EDTA, and 133 μmol/L Suc-LLVY-AMC. Proteasome inhibition activity was measured at 37° C. by measuring fluorescence. Suc-LLVY-AMC was obtained from AnaSpec, Inc. (San Jose, Calif.).

IC₅₀ values were determined by plotting the peptide hydrolysis rates as a function of the inhibitor concentrations and fitting those data to the Hill form of a dose-response curve. This enzymatic assay has been successfully used to establish the activity of a rationally designed proteasome inhibitor as shown in examples herein. The same assay was used to asses human proteasome activity and to determine IC₅₀ values.

### Example 8

### Results of Enzymatic Assay to Assess Proteasome Inhibition

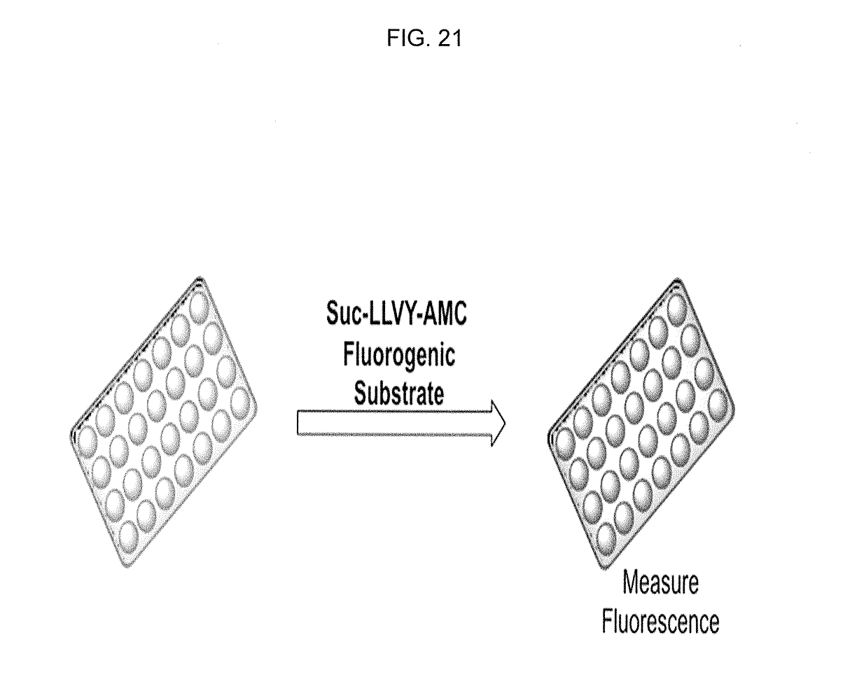

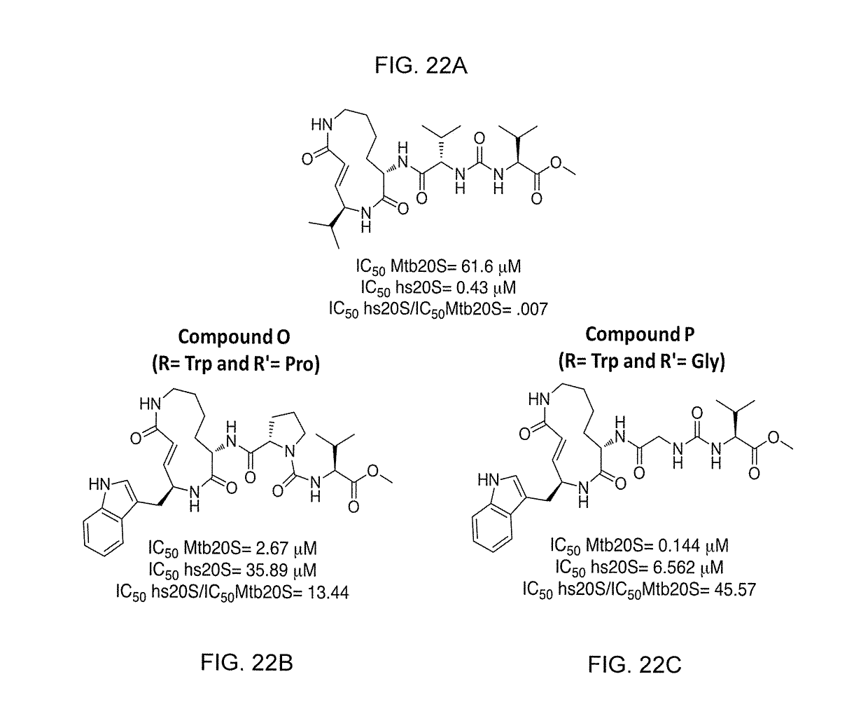

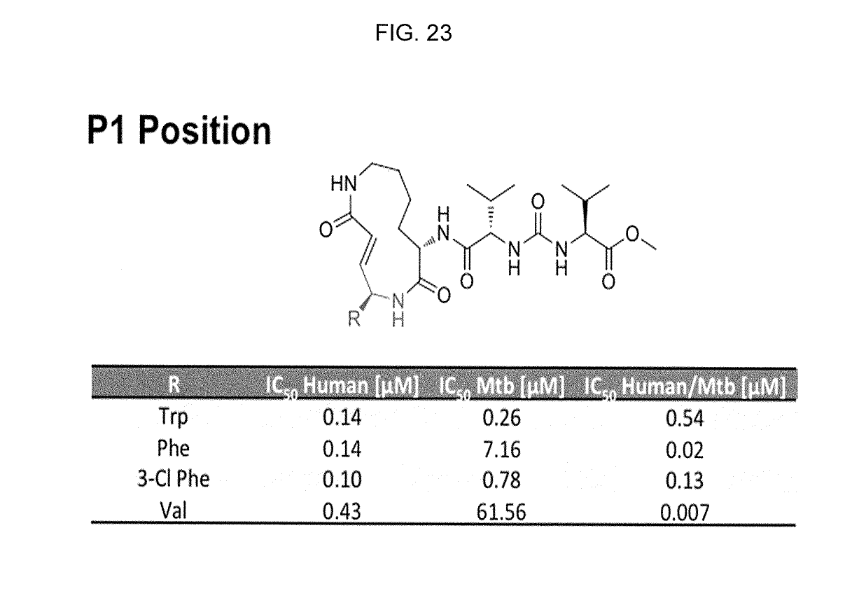

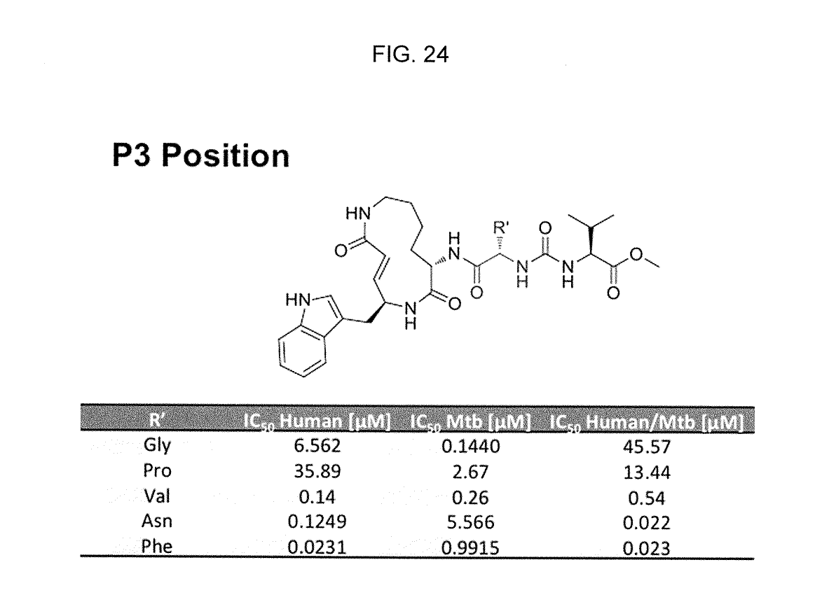

An overview of proteasome inhibition assay results for Syringolin B, Compound O and Compound P is shown in FIG. 22A-FIG. 22C. The ratio of IC₅₀ values for inhibition of M. tuberculosis and Human proteasome by Syringolin B indicates that Syringolin B does not selectively inhibit M. tuberculosis proteasome compared to inhibition of human proteasome (FIG. 22A). The ratio of IC₅₀ values for inhibition of M. tuberculosis and Human proteasome by Syringolin B analog Compound O indicates that Compound O selectively inhibits M. tuberculosis proteasome compared to human proteasome. The selectivity of proteasome inhibition by Compound O is more than 1,900 times improved compared to Syringolin B (FIG. 22B). The ratio of IC₅₀ values for inhibition of M. tuberculosis and Human proteasome by Syringolin B analog Compound P indicates that Compound P selectively inhibits M. tuberculosis proteasome compared to human proteasome (FIG. 22C). The selectivity of proteasome inhibition by Compound P is more than 6,500 times better than Syringolin B and more than three times better than Compound O. Compound P inhibits M. tuberculosis proteasome about three and half times more selectively than Compound O.

### Example 9

### Structure Activity Relationship (SAR) Analysis of Syringolin B Analogs

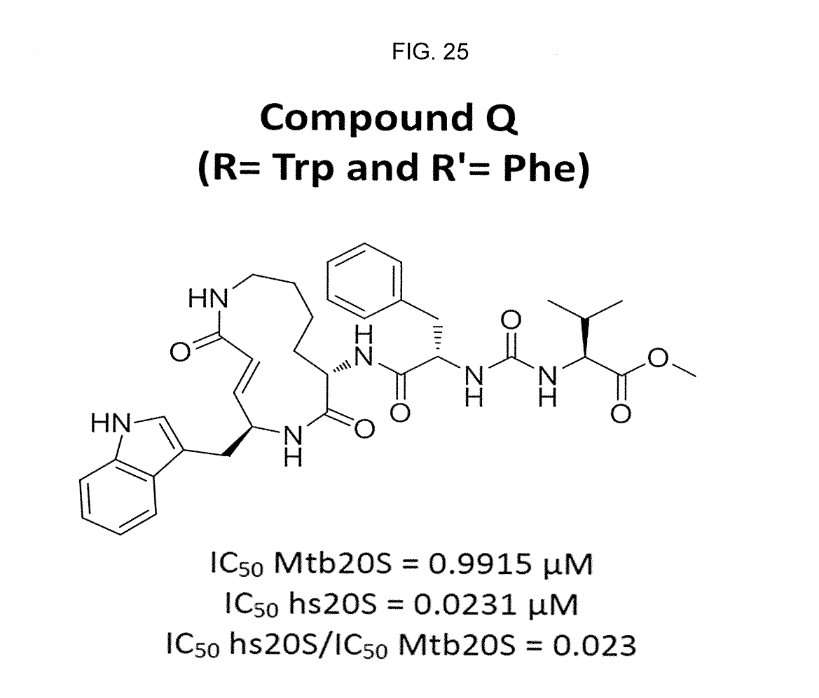

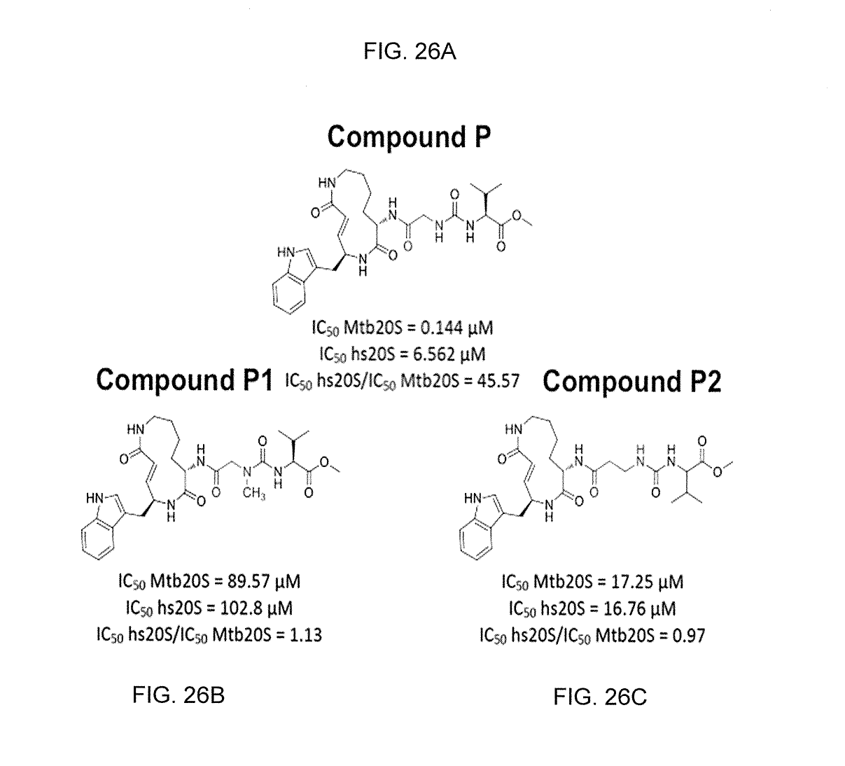

The Syringolin B analogs, Compounds O and P were evaluated and analyzed for Structure Activity Relationship (SAR). The SAR analysis (FIGS. 23 and 24) shows key structural features that impact the biological activity of Compounds O and P. The R position residue of Syringolin B analog faintly impacts the proteasome inhibition selectivity (FIG. 23).

Four amino acid residues were tested at the R position of Syringolin B analog, Tryptophan, Phenylalanine, 3-Cl Phenylalanine and Valine. Among the four residues analyzed at the R position it was observed that Tryptophan residue positively impacts proteasome inhibition selectivity. Syringolin analogs with Tryptophan at R position selectively inhibit M. tuberculosis proteasome about 77 times more than Syringolin analogs with Valine at R position.

The residue at the R′ position of Syringolin B analog is most critical for determining proteasome inhibition species selectivity (FIG. 24). Five amino acids were tested at the R′ position of the Syringolin analogs: Glycine, Proline, Valine, Aspargine and Phenylalanine. Among the five residues tested and analyzed at the R′ position it was observed that Glycine residue positively impacted proteasome inhibition selectivity. Syringolin analogs with Glycine at R′ position selectively inhibited M. tuberculosis proteasome about 1,981 times more than Syringolin analogs with Phenylalanine at the R′ position.

### Example 10

### SAR Analysis of Compound P

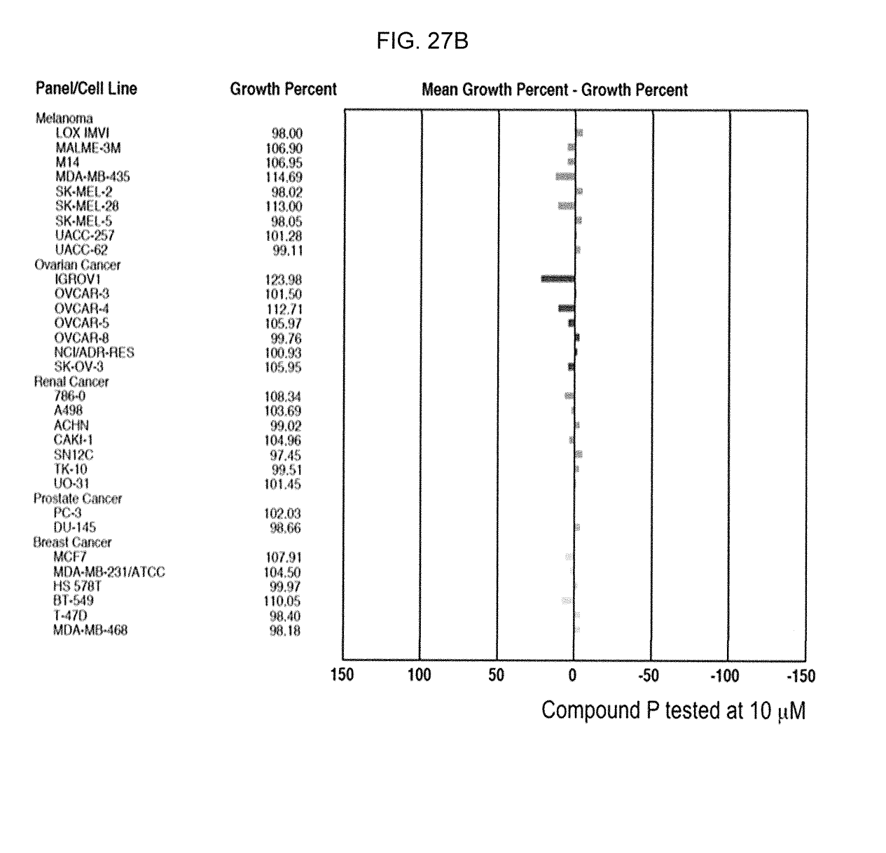

Compound P and its analogs were evaluated and analyzed for Structure Activity Relationship (SAR) as shown in FIG. 26A-FIG. 26C. Compound P is a Syringolin B analog with glycine at position R′ (FIG. 26A). Two variants of Compound P were designed and tested. FIG. 26B shows Compound P1 in which glycine is substituted with a glycine analog, which contains a methyl group that replaces Hydrogen. The IC50 values of Compound P1 indicate that the P1 analog does not inhibit M. tuberculosis proteasome or human proteasome. FIG. 26C shows Compound P2 in which the glycine is substituted with a glycine analog with extra carbons. The IC₅₀ value of Compound P2 indicates that this analog does not inhibit M. tuberculosis proteasome or human proteasome. Therefore, presence of glycine at R′ position is essential for proteasome inhibition as Compound P analogs Compounds P1 and P2 with glycine analogs were observed to be inactive.

### Example 11

### SAR Analysis of Compound Q

Compound Q is an analog of Syringolin B. Compound Q was prepared by diversity-oriented synthesis as described in examples herein as a negative control to Compound P. As described in examples and drawings herein the glycine residue at position R′ of Compound P is critical for inhibition of M. tuberculosis proteasome. In Compound Q the critical glycine residue at R′ position was replaced with phenylalanine, thereby designing and synthesizing a negative control compound which would not inhibit M. tuberculosis proteasome.

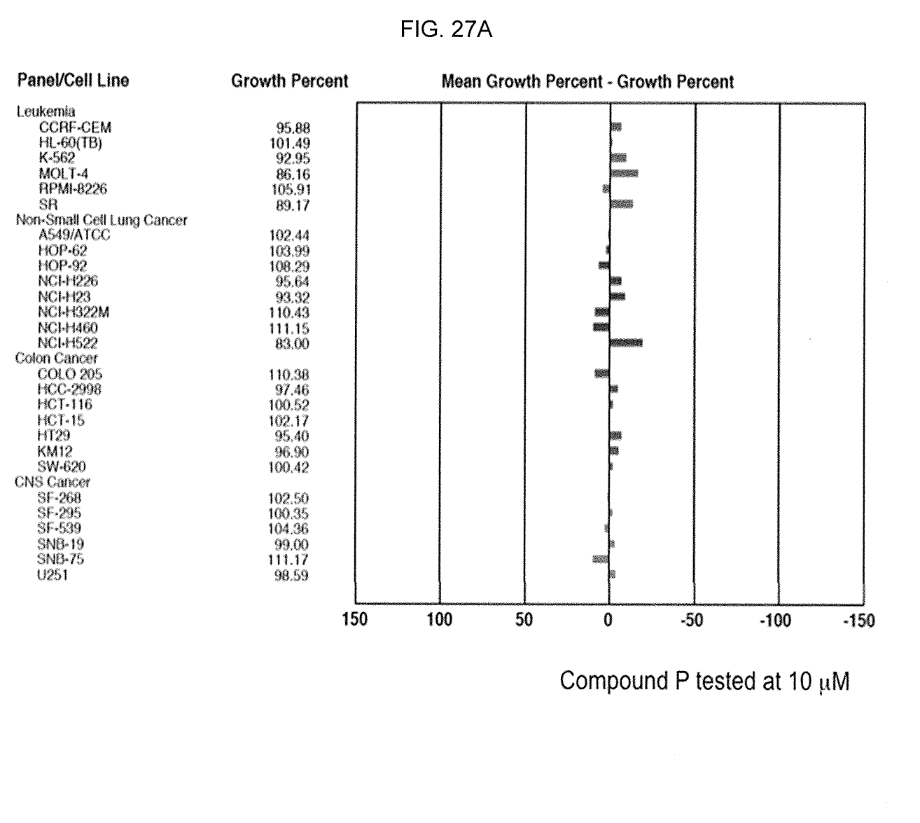

The half maximal inhibitory concentrations (IC₅₀) were calculated for Compound Q by enzymatic assay as described in examples herein. The IC₅₀ data shown in FIG. 25 indicate that Compound Q does not inhibit M. tuberculosis proteasome.

### Example 12

### Bioactivities of Compound P Against Sixty Cancer Cell Lines

The In Vitro Cell Line Screening Project (IVCLSP) is a dedicated service providing direct support to the DTP anticancer drug discovery program. The screen utilizes 60 different human tumor cell lines, representing leukemia, melanoma and cancers of the lung, colon, brain, ovary, breast, prostate, and kidney. IVCLSP is unique because the complexity of a 60 cell line dose response produced by a given compound results in a biological response pattern, which can be utilized in pattern recognition algorithms. Using the pattern recognition algorithms, it is possible to assign a putative mechanism of action to a test compound, or to determine that the response pattern is unique and not similar to that of any of the standard prototype compounds included in the NCI database. The methodology of the in vitro cancer screen is available from the NIH.

Syringolin B and Compound P were tested with NIH's IVCLSP program and data was obtained from 60 cancer cell lines. The data obtained from the screen in shown in FIG. 27A-FIG. 27B. The data indicate that Compound P does not inhibit cancer cell proteasomes and as a result does not kill cancer cells. Therefore, Compound P selectively inhibits bacterial proteasomes such as M. tuberculosis, S. coelicolor, and M. bovis BCG. As Compound P does not inhibit human proteasome, it is inferred that he compound is not toxic to human cells.

FIG. 27C shows an IVCLSP screen data comparison of Syringolin B and Compound P. The data obtained from IVCLSP is consistent with the data obtained herein from in vitro enzymatic assay. Syringolin B having valine at both R and R′ position is 144 times more selective for human proteasome, and Compound P having tryptophan at R position and Glycine at R′ position is 46 times more selective for M. tuberculosis proteasome.

A skilled person will recognize that many suitable variations of the compositions and methods are possible in addition to those described above and in the claims. It should be understood that the implementation of other variations and modifications of the embodiments of the invention and its various aspects will be apparent to one skilled in the art, and that the invention is not limited by the specific embodiments described herein and in the claims. Therefore, it is contemplated to cover the present embodiments of the invention and any and all modifications, variations, or equivalents that fall within the true spirit and scope of the basic underlying principles disclosed and claimed herein.

1. A compound having the formula: 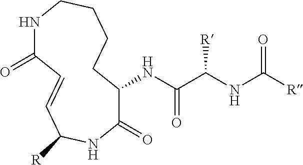
wherein:
R is covalently attached to a macrocycle and selected from the group consisting of benzyl, alkyl substituted benzyl, halogen substituted benzyl, CF₃ substituted benzyl, indole, CH₂-indole, pyrrole, CH₂-pyrrole, imidazole, CH₂-imidazole, pyrazole, CH₂-pyrazole, pyridine, CH₂-pyridine, pyrazine, CH₂-pyrazine, pyrimidine, CH₂-pyrimidine, pyridazine, CH₂-pyridazine, heterocyclic aromatic, fused aromatic with a linked CH₂, and a fused aromatic without a linked CH₂;
R′ is selected from the group consisting of Hydrogen, alkyl, alkyne, alkane, alkene, benzyl, alkyl substituted benzyl, halogen substituted benzyl, CF₃ substituted benzyl, indole, CH₂-indole, pyrrole, CH₂-pyrrole, imidazole, CH₂-imidazole, pyrazole, CH₂-pyrazole, pyridine, CH₂-pyridine, pyrazine, CH₂-pyrazine, pyrimidine, CH₂-pyrimidine, pyridazine, CH₂-pyridazine, heterocyclic aromatic, fused aromatic with a linked CH₂, fused aromatic without a linked CH₂ isopropyl, CH₂CH(CH₃)₂, CH(CH₃)CH₂CH₃, CH₂CH₂—S—CH₃, CH₂SH, arginine side chain, and lysine side chain; and,
R″ is a substituted urea or an amide and the substitution is selected from the group consisting of an alkyl, an amine, an aryl, an amino acid, and a fatty acid.

2. The compound according to claim 1, wherein the macrocycle ring comprises an additional unsaturated carbon-carbon bond.

3. The compound according to claim 1, wherein R″ is a substituted amino acid.

4. The compound according to claim 3, wherein R″ is NHC(CH₃)₂COOR¹.

5. The compound according to claim 4, wherein R¹ is selected fiom the group consisting of methyl, ethyl, isopropyl and tertiary butyl.

6. The compound according to claim 4, wherein R¹ is a fused aromatic and is selected from the group consisting of: pentalene, indene, naphthalene, azulene, as-indacene, s-indacene, biphenylene, acenapthylene, fluorene, phenalene, heptalene, and phenanthrene.

7. The compound according to claim 1, wherein R″ is an alkyl substituted amine of length C₅-C₁₅.

8. The compound according to claim 7, wherein the alkyl substituted amine is of length C₉-C₁₁.

9. A pharmaceutical composition comprising a compound according to claim 1 as an active ingredient and a pharmaceutically acceptable carrier, salt, or buffer.

10. A method for inhibiting proteasome activity in a cell comprising:
contacting the cell with a compound of the core formula 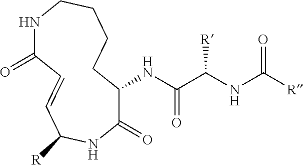
wherein:
R is covalently attached to a macrocycle and selected from the group consisting of benzyl, alkyl substituted benzyl, halogen substituted benzyl, CF₃ substituted benzyl, indole, CH₂-indole, pyrrole, CH₂-pyrrole, imidazole, CH₂-imidazole, pyrazole, CH₂-pyrazole, pyridine, CH₂-pyridine, pyrazine, CH₂-pyrazine, pyrimidine, CH₂-pyrimidine, pyridazine, CH₂-pyridazine, heterocyclic aromatic, fused aromatic with a linked CH₂, and a fused aromatic without a linked CH₂;
R′ is selected from the group consisting of Hydrogen, alkyl, alkyne, alkane, alkene, benzyl, alkyl substituted benzyl, halogen substituted benzyl, CF₃ substituted benzyl, indole, CH₂-indole, pyrrole, CH₂-pyrrole, imidazole, CH₂-imidazole, pyrazole, CH₂-pyrazole, pyridine, CH2-pyridine, pyrazine, CH₂-pyrazine, pyrimidine, CH₂-pyrimidine, pyridazine, CH₂-pyridazine, heterocyclic aromatic, fused aromatic with a linked CH₂, fused aromatic without a linked CH₂ isopropyl, CH₂CH(CH₃)₂, CH(CH₃)CH₂CH₃, CH₂CH₂—S—CH₃, CH₂SH, arginine side chain, and lysine side chain; and,
R″ is a substituted urea or an amide and the substitution is selected from the group consisting of an alkyl, an amine, an aryl, an amino acid, and a fatty acid,
analyzing amount of inhibition of the proteasome activity of the cell.

11. The method according to claim 10, wherein the macrocycle ring comprises an additional unsaturated carbon-carbon bond.

12. The method according to claim 10, wherein R″ is a substituted amino acid.

13. The method according to claim 12, wherein R″ is NHC(CH₃)₂COOR¹.

14. The method according to claim 13, wherein R¹ is selected from the group consisting of methyl, ethyl, isopropyl and tertiary butyl.

15. The method according to claim 14, wherein R¹ is a fused aromatic and is selected from the group consisting of: pentalene, indene, naphthalene, azulene, as-indacene, s-indacene, biphenylene, acenapthylene, fluorene, phenalene, heptalene, and phenanthrene.

16. The method according to claim 10, wherein the cell is a bacterial cell.

17. The method according to claim 10, wherein the cell is a eukaryotic cell.

18. The method according to claim 10, wherein analyzing inhibition of the proteasome activity comprises detecting inhibition of at least one type of proteasome activity selected from caspase-like activity, trypsin-like activity, and chymotrypsin-like activity.

19. The method according to claim 18, wherein detecting inhibition of the at least one type of proteasome activity is performed using a small molecule substrate for the respective proteasome activity.

20. The method for inhibiting proteasome activity in a cell according to claim 10, wherein R″ is an alkyl substituted amine of length C₅-C₁₅.

21. The method according to claim 10, wherein analyzing further comprises observing inhibition of proteasome activity in a Mycobacterium tuberculosis cell at an amount which is greater than an amount of inhibition of a human cell proteasome activity by at least: about 2-fold, about 5-fold, about 10-fold, about 20-fold, about 30-fold, about 40-fold, or about 50-fold.

22. The method according to claim 10, wherein analyzing further comprises observing inhibition of proteasome activity in a Streptomyces coelicolor cell at an amount which is greater than an amount of inhibition of a human cell proteasome activity by at least: about 2-fold, about 5-fold, about 10-fold, about 20-fold, about 30-fold, about 40-fold, or about 50-fold, wherein the Streptomyces coelicolor cell is a surrogate for a Mycobacterium tuberculosis cell.

23. A method for screening analogs or derivatives of a syringolin to identify compounds which inhibit an actinobacterial proteasome activity, the method comprising:
culturing indicator cells with the analogs or derivatives of syringolin; and,
isolating and measuring amount of mRNA of protein non-heme chloroperoxidase, wherein an increase in the amount of the non-heme chloroperodixase mRNA indicates inhibition of proteasome activity in the cells.

24. The method according to claim 23, wherein the non-heme chloroperoxidase is SCO0465.

25. The method according to claim 23, wherein the indicator cells are selected from the group of Streptomyces coelicolor, S. griseus, S. rimosus, S. clavuligerus, S. alboniger, S. venezuelae, S. avermitilis, S. fradiae, S. lincolnensis, S. roseosporus, S. platensis, and S. verticillus.

26. A method of treating a mammalian subject for infection by a bacterial pathogen, the method comprising:
administering to the subject a compound of the core formula: 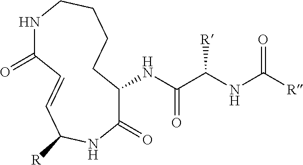
in an amount effective for inhibiting proteasome activity by the pathogen in the subject wherein:
R is covalently attached to a macrocycle and selected from the group consisting of benzyl, alkyl substituted benzyl, halogen substituted benzyl, CF₃ substituted benzyl, indole, CH₂-indole, pyrrole, CH₂-pyrrole, imidazole, CH2-imidazole, pyrazole, CH₂-pyrazole, pyridine, CH₂-pyridine, pyrazine, CH₂-pyrazine, pyrimidine, CH₂-pyrimidine, pyridazine, CH2-pyridazine, heterocyclic aromatic, fused aromatic with a linked CH₂, and a fused aromatic without a linked CH₂;
R′ is selected from the group consisting of Hydrogen, alkyl, alkyne, alkane, alkene, benzyl, alkyl substituted benzyl, halogen substituted benzyl, CF₃ substituted benzyl, indole, CH₂-indole, pyrrole, CH₂-pyrrole, imidazole, CH₂-imidazole, pyrazole, CH₂-pyrazole, pyridine, CH₂-pyridine, pyrazine, CH₂-pyrazine, pyrimidine, CH₂-pyrimidine, pyridazine, CH₂-pyridazine, heterocyclic aromatic, fused aromatic with a linked CH₂, fused aromatic without a linked CH₂ isopropyl, CH₂CH(CH₃)₂, CH(CH₃)CH₂CH₃, CH₂CH₂—S—CH₃, CH₂SH, arginine side chain, and lysine side chain; and,
R″ is a substituted urea or an amide and the substitution is selected from the group consisting of an alkyl, an amine, an aryl, an amino acid, and a fatty acid.

27. The method according to claim 26, wherein the macrocycle ring comprises an additional unsaturated carbon-carbon bond.

28. The method according to claim 26, wherein R″ is a substituted amino acid.

29. The method according to claim 28, wherein R″ is NHC(CH₃)₂COOR¹.

30. The method according to claim 29, wherein R¹ is selected from the group consisting of methyl, ethyl, isopropyl and tertiary butyl.

31. The method according to claim 29, wherein R¹ is a fused aromatic and is selected from the group consisting of: pentalene, indene, naphthalene, azulene, as-indacene, s-indacene, biphenylene, acenapthylene, fluorene, phenalene, heptalene, and phenanthrene.

32. The method according to claim 26, wherein R″ is an alkyl substituted amine of length C₅-C₁₅.

33. The method according to claim 26, further comprising treating the subject for the pathogen which is selected from the group consisting of M. tuberculosis, M. leprae, M. avium, M. avium paratuberculosis, Nocardia cyriacigeorgica, N. farcinica, N. abscessus, N. asteroides, N. brasiliensis, N. nova, N. otitidiscaviarum, N. paucivorans, N. pseudobrasiliensis, N. transvalensis, N. veteran, N. wallacei, N. africana, N. anaemiae, N. araoensis, N. arthritidis, N. asiatica, N. beijingensis, N. blacklockiae, N. brevicatena, N. carnea, N. concavca, N. corynebacteroides, N. elegans, N. exalbida, N. higoensis, N. ignorata, N. inohanensis, Corynebacterium pseudotuberculosis, C. renale, C. cystidis, C. pilosum, C. diphtheria and C. bovis.

34. The method according to claim 26, wherein inhibiting proteasome activity in the subject further comprises measuring inhibition of proteasome activity of M. tuberculosis in the subject, thereby inhibiting survival of the M. tuberculosis under the conditions of nitrooxidative stress that exist in vivo during infection.

35. The method according to claim 26, further comprising administering to the subject an additional therapeutic agent.

36. The method according to claim 35, wherein the additional therapeutic agent is selected from the group of: isoniazid, rifampin, ethambutol, pyrazinamide, ethionamide, cycloserine, p-aminosalicyclic acid, clofazimine, amoxicillin/clavulanic acid, clarithromycin, rifabutin, thiacetazone, fluoroquinolone, and aminoglycoside.
